(19) United States
(12) Patent Application Publication
(10) Pub. No.: US 2010/0003164 A1
(43) Pub. Date: Jan. 7, 2010

(54) SYSTEMS AND METHODS FOR MANAGING AIR QUALITY

(75) Inventors: Neil Bourne, Sydney (AU); Bob Greenfield, Sydney (AU)

Correspondence Address: BAINWOOD HUANG & ASSOCIATES LLC, 2 CONNECTOR ROAD, WESTBOROUGH, MA, 01581, US

(73) Assignee: MICROGENIX AUSTRALASIA PTY LIMITED, Sydney, NSW (AU)

(21) Appl. No.: 12/375,717

(22) PCT Filed: Jul. 30, 2007
(86) PCT No.: PCT/AU2007/001056

§ 371 Date: May 15, 2009

(30) Foreign Application Priority Data
Jul. 31, 2006 (AU) 2006904176
Sep. 12, 2006 (AU) 2006905031

(51) Int. Cl. A61L 9/22; A61L 9/00

(52) U.S. Cl. 422/4; 422/121; 422/120

(57) ABSTRACT

A system for managing air quality according to the present invention includes an air processing unit, said unit including an inlet for receiving feed air from a unidirectional air passage. A treatment path housing a module is coupled to inlet for receiving, processing and passing the feed air to remove one of more contaminants. Said module includes a body and one or more UV tubes in the body for emitting UV light at an ozone production wavelength and/an ozone destruction wavelength. Feed air travels through module housed in said treatment path. An outlet is couple to said treatment path for passing the feed air to the passage at a location downstream of inlet. The pressure drop between the inlet and the outlet is less than a predetermined value.

### FIELD OF THE INVENTION

The present invention relates to air quality management, and more particularly to systems and methods for managing air quality.

Embodiments of the invention have been developed to provide cost effective, energy efficient, and relatively safe approaches for managing issues of air quality for indoor human environments in a way that addresses concerns such as bioterrorism and employee protection. Although the invention is described hereinafter with reference to applications such as these, it will be appreciated that the invention is applicable in broader contexts.

### BACKGROUND

Air quality management is a growing concern, particularly in indoor environments where recirculated air accounts for upwards of 50% of the air present. That is, for an average sample of air within the environment, 50% or more of that air has been drawn from within the indoor environment, subjected to some processing, and re-released into the indoor environment. The remainder of the air had been drawn from an external environment. It is common in environments such as high-rise buildings or large ships for recirculated air to account for 70% to 85% of air present.

There are typically two major competing considerations to be addressed when developing and implementing and air quality management solution: the need to maintain air quality at acceptable levels for human wellbeing and comfort, and the desire to operate in a cost and energy effective manner.

There is a need for systems and methods that allow for the management of air quality in a manner to balance these and other considerations.

### SUMMARY

According to a first aspect of the invention, there is provided a system for managing air quality including:

an inlet for receiving feed air from a unidirectional air passage at a flow rate;

a treatment path coupled to the inlet for receiving, processing and passing the feed air to remove one or more air contaminants;

an outlet coupled to the treatment path for receiving the feed air from the treatment path and passing the feed air to the unidirectional air passage at a location downstream of the input, wherein the pressure drop between the inlet and outlet is less than a predetermined value for a given flow rate.

According to one embodiment the treatment path includes at least one chamber having a UV light source for processing the feed air.

According to one embodiment one of the at least one chambers includes a UV light source that is optimized for air sterilization.

According to one embodiment at least one of the chambers includes a UV light source that is optimized for odor removal.

According to one embodiment the treatment path includes:

an ozone production section for converting at least some oxygen molecules in the feed air into ozone molecules; and

an ozone destruction section for converting at least some of the ozone molecules into oxygen molecules.

According to one embodiment the ozone production section includes a first chamber having a first UV light source for emitting UV light at an ozone production wavelength for converting at least some oxygen molecules in the feed air into ozone molecules.

According to one embodiment the ozone destruction includes a second chamber downstream of and coupled to the first chamber having a second UV light source for emitting UV light at a ozone destruction wavelength for converting at least some of the ozone molecules into oxygen molecules.

According to one embodiment the ozone production section and the ozone destruction section are defined by a single chamber.

According to one embodiment the single chamber includes a single UV light source having a first portion for emitting UV light at an ozone production wavelength for converting at least some oxygen molecules in the feed air into ozone molecules and a second downstream portion for emitting UV light at a ozone destruction wavelength for converting at least some of the ozone molecules into oxygen molecules.

According to one embodiment the treatment path includes a particulate filter for preventing passage of particles of greater than a threshold size.

According to one embodiment the threshold size is 100 microns.

According to one embodiment the threshold size is 10 microns.

According to one embodiment the particulate filter is provided upstream of an antimicrobial filter.

According to one embodiment the treatment path includes an antimicrobial filter for reducing pathogens levels in the feed.

According to one embodiment the antimicrobial filter has a pathogen reduction rate of greater than 60% for pathogens sized between 0.01 microns and 100 microns.

According to one embodiment the antimicrobial filter has a pathogen reduction rate of greater than 60% for pathogens sized between 0.01 microns and 0.3 microns.

According to one embodiment the antimicrobial filter has a pathogen reduction rate of greater than 80% for pathogens sized between 0.01 microns and 100 microns.

According to one embodiment the antimicrobial filter has a pathogen reduction rate of greater than 80% for pathogens sized between 0.01 microns and 0.3 microns.

According to one embodiment the antimicrobial filter has a pathogen reduction rate of between 60% and 95% for pathogens sized between 0.01 microns and 100 microns.

According to one embodiment the antimicrobial filter has a pathogen reduction rate of between 60% and 95% for pathogens sized between 0.01 microns and 0.3 microns.

According to one embodiment the antimicrobial filter includes as an active ingredient at least one quaternary ammonium salt.

According to one embodiment the at least one salt includes either or both of a chloride or bromide salt.

According to one embodiment at least one salt includes as a substitute to a nitrogen atom a silane group.

According to one embodiment the silane group is a trialkyloxysilane group.

According to one embodiment the silane group is a trimethyloxysilane group.

According to one embodiment the system includes an air turbulation device upstream of at least one of the UV light sources.

According to one embodiment the air turbulation device includes an impeller or propeller.

According to one embodiment the air turbulation device includes a fixed air-guiding formation.

According to one embodiment the treatment path includes an antimicrobial filter upstream of one or more chambers having respective UV light sources.

According to one embodiment the treatment path includes a particulate filter upstream of the antimicrobial filter.

According to one embodiment a plurality of the chambers are longitudinally spaced in the treatment path.

According to one embodiment a plurality of the chambers are laterally spaced in the treatment path.

According to one embodiment at least one of the chambers includes an UV light source including a UV tube disposed substantially parallel to the treatment path.

According to one embodiment at least one of the chambers includes an UV light source including a UV tube disposed substantially perpendicular to the treatment path.

According to one embodiment at least one of the chambers includes an UV light source including a UV tube having a first portion for emitting a first wavelength of UV light and a second portion for emitting a second wavelength of UV light.

According to one embodiment the first and second wavelengths are selected such that at least some oxygen molecules are converted to ozone molecules in an upstream region of the chamber and at leas some of the ozone molecules are converted to oxygen molecules in a downstream region of the chamber.

According to one embodiment the first portion is longitudinally spaced from the second portion.

According to one embodiment the first portion is circumferentially spaced from the second portion.

According to one embodiment the predetermined value is a pressure drop of between 30 Pa to 90 Pa for a flow rate of about 3 linear meters per second.

According to one embodiment the predetermined value is a pressure drop of between 50 Pa to 75 Pa for a flow rate of about 3 linear meters per second.

According to one embodiment the treatment path provides a substantially linear pathogen reduction rate across the spectrum of pathogens sized between 0.01 and 0.03 microns.

According to one embodiment the treatment path provides a substantially linear pathogen reduction rate across the spectrum of pathogens sized between 0.01 and 100 microns.

According to one embodiment the treatment path includes:

a first section including a antimicrobial filter for performing a primary pathogen reduction functionality;

a second section including at least one UV light source for performing a secondary pathogen reduction functionality, wherein the UV light source further facilitates an ozonation process for odor reduction.

According to one embodiment the UV light operates at either or both of an ozone production wavelength and ozone destruction wavelength.

According to one embodiment the processing along the treatment path is substantially ozone negative.

According to one embodiment the predetermined value is a pressure drop of between 30 Pa to 90 Pa for a flow rate of about 3 linear meters per second and the effective pathogen reduction rate along the treatment path is above 65%.

According to one embodiment the system includes one or more sensors for measuring one or more air characteristics in or adjacent the treatment path.

According to one embodiment the one or more characteristics include any of odor levels, pathogen levels, particulate levels, flow rate, contaminant levels, and ozone levels.

According to one embodiment the one or more sensors are coupled to a central controller.

According to one embodiment the central controller is responsive to one or more of the measured air characteristics for selectively providing a signal.

According to one embodiment the central controller is further responsive to data indicative of one or more system operational characteristics for selectively providing a signal.

According to one embodiment the central controller is further responsive to data indicative of one or more external factors for selectively providing a signal.

According to one embodiment the signal is indicative of a command to adjust one or more system operational characteristics.

According to one embodiment the air treatment path includes:

a first chamber having a first UV light source for emitting UV light at an ozone production wavelength for converting at least some oxygen molecules in the feed air into ozone molecules; and

a second chamber downstream of the first chamber having a second UV light source for emitting UV light at a ozone destruction wavelength for converting at least some of the ozone molecules into oxygen molecules; and

wherein the signal is indicative of a command to adjust one or more light emission characteristics of either or both of the first and second sources to reduce the risk of greater than a threshold level of ozone being provided downstream of the second chamber.

According to one embodiment one of the sensors is provided downstream of the second chamber for measuring the ozone content of air downstream of the second chamber and the central controller provides the signal in response to the ozone content reaching a predetermined value.

According to one embodiment the central controller selectively provides the signal in response to a receiving data indicative change one or more light emission characteristics.

According to one embodiment one of the sensors is provided upstream of the first chamber for measuring odor levels of air upstream of the first chamber and the central controller provides the signal in response to a threshold variation in the odor levels.

According to one embodiment the command is an instruction to either increase or reduce light emission in either or both of the first and second chambers.

According to one embodiment increasing or reducing light emission includes respectively activating or deactivating one or more UV producing tubes.

According a second aspect of the invention there is provided a system for managing air quality including:

a first chamber including an inlet for receiving a continuous feed of air flow including oxygen gas molecules and an outlet for allowing continuous passage of the air flow through the fist chamber;

a first UV light source located in the first chamber for converting at least some of the oxygen gas molecules into ozone molecules; and

a second chamber including a second inlet coupled to the first outlet for receiving air flow from the first chamber and an second outlet for allowing continuous passage of the air flow through the second chamber;

a second UV light source located in the second chamber for converting at least some of the ozone molecules into oxygen gas molecules;

at least one sensor for measuring one or more properties of the air flow at one or more of the first input, second input and second output;

a controller responsive to one or more of the measured air properties for selectively adjusting one or more operational characteristics of either or both of the first and second UV light sources.

According to a third aspect of the invention there is provided a system for managing air quality including:

an inlet for receiving a continuous flow of feed air;

a first outlet for providing the feed air to an NBC filtration system upstream of a system according to the first aspect;

a second outlet for providing the feed air to a location downstream of the NBC filtration system but upstream of the system according to the first aspect; and

a control unit responsive to a command for selecting between the first and second outlets.

According to a fourth aspect of the invention there is provided a method for managing air quality including the steps of:

providing feed air to a location downstream of an NBC filtration system but upstream of a system according to the first aspect; and

being responsive to a command for providing the feed air to the NBC filtration system.

According to a fifth aspect of the invention there is provided a system for managing air quality including:

a structure including at least one upwardly extending member for extending from a datum and supporting at least one outwardly extending member, the space between the upwardly extending member and the outwardly extending member at least partially defining a target zone for containing one or more persons;

an inlet formed in the upwardly extending member for substantially outwardly drawing sample air from the target zone;

an air processing unit coupled to the inlet for processing the sample air to produce baseline air having predefined characteristics;

an outlet formed in the outwardly extending member and coupled to the air processing unit for substantially downwardly providing the baseline air to the target zone.

According to one embodiment the provision of baseline air to the target zone provides an air curtain of sample air to the target zone.

According to one embodiment the inlet is adapted to draw sample air including at least a portion of exhaled air from the one or more persons.

According to one embodiment the system includes a sensor intermediate the inlet and the air processing unit for measuring one or more air characteristics.

According to one embodiment the system includes a processor coupled to the sensor for comparing the one or more measured characteristics with the predefined characteristics and selectively providing a signal indicative of a variation between the one or more measured characteristics from the predefined characteristics.

According to one embodiment the inlet includes an intake aperture formed in the upwardly extending member at a height of between 1 meter and 2.5 meters from the datum.

According to a sixth aspect of the invention there is provided a method for managing air quality including the steps of:

directing a person to stand within the target zone of a system according to the fifth aspect;

for a predetermined time measuring one or more characteristics of the sample air;

comparing the one or more measured characteristics with the predefined characteristics; and

selectively providing a signal indicative of a variation between the one or more measured characteristics from the predefined characteristics.

According to one embodiment the method includes a preliminary step of conducting a preliminary analysis of one or more aspects of the person and responsive to that analysis providing a signal for directing the person to stand within the target zone.

According to one embodiment the preliminary analysis includes a thermal imaging technique.

According to one embodiment the preliminary analysis includes an assessment of the person's history.

According to a seventh aspect of the invention there is provided a device for managing air quality in combination with a fan coil unit having a circulation module for drawing air from a first region, passing the air through a cooling zone, and subsequently providing the air to a second region, the device including:

a body insertable into the fan coil unit upstream of the cooling zone;

an inlet on the body for receiving air drawn by the circulation module from the first region;

an ozone production zone within the body downstream of the inlet;

an ozone destruction zone within the body downstream of the ozone production zone;

an outlet on the body downstream of the ozone destruction zone for allowing passage of the drawn air to the cooling zone;

wherein the pressure drop between the inlet and the outlet is less than a predetermined value.

In one embodiment the predetermined value is about 150 Pa.

In one embodiment the body is adapted to fit in a void substantially adjacent the upstream end of the cooling zone.

In one embodiment the outlet is formed on a side of the body that is configured for conformity with an adjacent upstream end of the cooling zone.

In one embodiment the body is substantially shaped as a triangular prism.

In one embodiment the body is substantially shaped as a trapezoidal prism.

In one embodiment the device includes a mounting array for secure connection to the fan coil unit such that the device is slidably mounted with respect to the fan coil unit.

In one embodiment the device includes one or more filters upstream of the ozone production zone.

In one embodiment the one or more filters include a particulate filter.

In one embodiment the one or more filters include an antimicrobial filter.

In one embodiment the one or more filters include a particulate filter and a downstream antimicrobial filter.

According to a seventh aspect of the invention, there is provided a method for managing air quality, the method including the steps of:

identifying a fan coil unit having a circulation module for drawing air from a first region, passing the air through a cooling zone, and subsequently providing the air to a second region;

inserting into the fan-coil unit upstream of the cooling zone a device for managing air quality according to any one of claims 73 to 83.

According to an eighth aspect of the invention, there is provided a device for managing air quality, the device including:

a body for supporting a rooftop air processing unit, the unit having a circulation module for drawing a predetermined combination of first air from a building and second air from an outside environment, passing the first and second air through a cooling zone, and subsequently providing to the building third air defined by the first and second air;

one or more apertures formed in the body for allowing airflow communication between the building and the air processing unit for the drawing of first air and provision of second air;

a structure on the body for mounting an air processing device having an ozone production zone upstream of an ozone destruction zone such that the third air provided by the circulation module passes through the ozone production and ozone destruction zones intermediate the unit and the building.

In one embodiment the structure is slidably mounted with respect to the body such that outward sliding of the structure with respect to the body provides external access to the air processing device.

According to a further aspect of the invention, there is provided a method for managing air quality, the method including the steps of:

identifying a rooftop air processing unit having a circulation module for drawing a predetermined combination of first air from a building and second air from an outside environment, passing the first and second air through a cooling zone, and subsequently providing to the building third air defined by the first and second air;

placing a smart curb intermediate the unit and the rooftop, the smart curb including a structure for mounting an air processing device having an ozone production zone upstream of an ozone destruction zone such that the third air provided by the circulation module passes through the ozone production and ozone destruction zones intermediate the unit and the building.

According to a further aspect of the invention, there is provided a module for use with a system for managing air quality, wherein the system includes an inlet for receiving feed air and an outlet downstream of the inlet for passing the feed air, the module including:

a body removably mountable to the system such that the feed air travels through the module intermediate the inlet and the outlet;

one or more UV tubes in the body for emitting UV light at an ozone production wavelength and/or an ozone destruction wavelength.

One embodiment provides a module wherein at least one of the UV tubes is configured to be disposed normal to the direction of airflow when the body is mounted to the system.

One embodiment provides a module including a plurality of vertically spaced UV tubes.

One embodiment provides a module wherein the one or more UV tubes include at least one dual purpose UV tube having a first portion coated to emit UV light at an ozone production wavelength and a second portion coated to emit UV light at an ozone destruction wavelength.

One embodiment provides a module wherein the portions are defined such that less UV light is emitted at the ozone production wavelength than at the ozone destruction wavelength.

One embodiment provides a module wherein the first and second portions are circumferential portions.

One embodiment provides a module wherein the first and second portions are longitudinal portions.

One embodiment provides a module wherein the one or more UV tubes include at least one ozone production tube for emitting UV light at an ozone production wavelength and at least one ozone destruction tube for emitting UV light at an ozone destruction wavelength.

One embodiment provides a module including a reflector intermediate the at least one ozone production tube and the at least one ozone destruction tube thereby to define a ozone production zone and an ozone destruction zone, wherein UV light from the at least one ozone destruction tube is substantially absent in the ozone production zone and UV light from the at least one ozone production tube is substantially absent in the ozone destruction zone.

One embodiment provides a module wherein the reflector is curved about the at least one ozone production tube.

One embodiment provides a module wherein the system is defined by an air conditioning unit having a cooling zone, and wherein the body is removably mountable intermediate the inlet and the cooling zone.

One embodiment provides a module wherein the air conditioning unit is a vertical furred-in stack type fan coil air conditioning unit.

One embodiment provides a module wherein the one or more UV tubes are replaceably mounted in the body.

A further aspect of the invention provides a method a modifying an air conditioning unit having an inlet upstream of a cooling zone, the method including the step of disposing a module according to claim 1 intermediate the inlet and the cooling zone.

A further aspect of the invention provides a system for managing air quality, the system including:

a frame for defining:
an inlet for receiving feed air from a unidirectional air passage at a flow rate;
a module housing region for removably housing one or more air processing modules, the module housing region being configured for removably housing at least one UV module for containing one or more UV tubes that emit UV light at an ozone production wavelength and/or an ozone destruction wavelength; and
an outlet coupled to the treatment path for receiving the feed air from the treatment path and passing the feed air to the unidirectional air passage at a location downstream of the input; and

a power supply for providing power to at least module upon housing of that module in the module housing region.

One embodiment provides a system wherein the module housing region is configured for removably housing at least one antimicrobial filter module.

One embodiment provides a system wherein the module housing region is configured for removably housing a plurality of UV modules.

One embodiment provides a system wherein at least two of the plurality of UV modules are vertically stacked with respect to one another.

One embodiment provides a system wherein at least one of the UV tubes is configured to be disposed normal to the direction of airflow between the inlet and outlet.

A further aspect of the invention provides a system for managing air quality including:

an inlet for receiving feed air from a unidirectional air passage at a flow rate;

a treatment path coupled to the inlet for receiving, processing and passing the feed air to remove one or more air contaminants;

an outlet coupled to the treatment path for receiving the feed air from the treatment path and passing the feed air to the unidirectional air passage at a location downstream of the input, wherein the pressure drop between the inlet and outlet is less than a predetermined value for a given flow rate.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Embodiments of the invention will now be described, by way of example only, with reference to the accompanying drawings in which:

FIG. 1 is a schematic side view of an air processing unit according to one embodiment;

FIG. 1A is a schematic side view of an air processing unit according to another embodiment;

FIG. 1B is a schematic side view of an air processing unit according to another embodiment;

FIG. 2 illustrates an exemplary air processing method;

FIG. 3 is a schematic side view of an air processing system according to one embodiment;

FIG. 4 is a schematic sectional side view of an air processing system according to another embodiment;

FIG. 4A is a schematic sectional side view of an air processing system according to another embodiment;

FIG. 5 is a schematic sectional end view of an air processing system according to another embodiment;

FIG. 6 is a schematic representation of an air processing system according to another embodiment;

FIG. 7 is a schematic representation of an air quality management system according to another embodiment;

FIG. 8 is a schematic sectional side view of a UV module according to another embodiment;

FIG. 8A is a schematic sectional side view of an air processing system according to another embodiment;

FIG. 9 is a schematic sectional side view of a UV module according to another embodiment;

FIG. 9A is a schematic sectional side view of an air processing system according to another embodiment;

FIG. 10 is a schematic sectional side view of a UV module according to another embodiment;

FIG. 10A is a schematic sectional side view of an air processing system according to another embodiment;

FIG. 10B is a schematic sectional side view of an air processing system according to another embodiment;

FIG. 11 is a schematic sectional side view of a UV module according to another embodiment;

FIG. 11A is a schematic sectional side view of an air processing system according to another embodiment;

FIG. 12 is a schematic sectional side view of a UV module according to another embodiment;

FIG. 12A is a schematic sectional side view of an air processing system according to another embodiment;

FIG. 12B is a schematic sectional side view of a UV module according to another embodiment;

FIG. 12C is a schematic sectional side view of an air processing system according to another embodiment;

FIG. 12D is a schematic top view of a UV tube assembly;

FIG. 13 is a schematic sectional side view of a UV module according to another embodiment;

FIG. 13A is a schematic sectional side view of an air processing system according to another embodiment;

FIG. 14 is a schematic sectional side view of a system for managing air quality according to a further embodiment; and

FIG. 15 is a schematic sectional side view of a system for managing air quality according to a further embodiment.

FIG. 16 is a schematic sectional side view of fan coil unit.

FIG. 16A is a schematic sectional side view of fan coil unit and a further embodiment of the invention.

FIG. 16B is a schematic sectional side view of fan coil unit in combination with a further embodiment of the invention.

FIG. 17 is a schematic sectional side view of fan coil unit in combination with a further embodiment of the invention.

FIG. 18 is a schematic sectional side view of fan coil unit.

FIG. 18A is a schematic sectional side view of fan coil unit and a further embodiment of the invention.

FIG. 18B is a schematic sectional side view of fan coil unit in combination with a further embodiment of the invention.

FIG. 19 is a schematic sectional side view of fan coil unit.

FIG. 19A is a schematic sectional side view of fan coil unit in combination with a further embodiment of the invention.

FIG. 20 is a schematic sectional side view of a rooftop air processing unit.

FIG. 20A is a schematic sectional side view of rooftop air processing unit in combination with a further embodiment of the invention.

### DETAILED DESCRIPTION

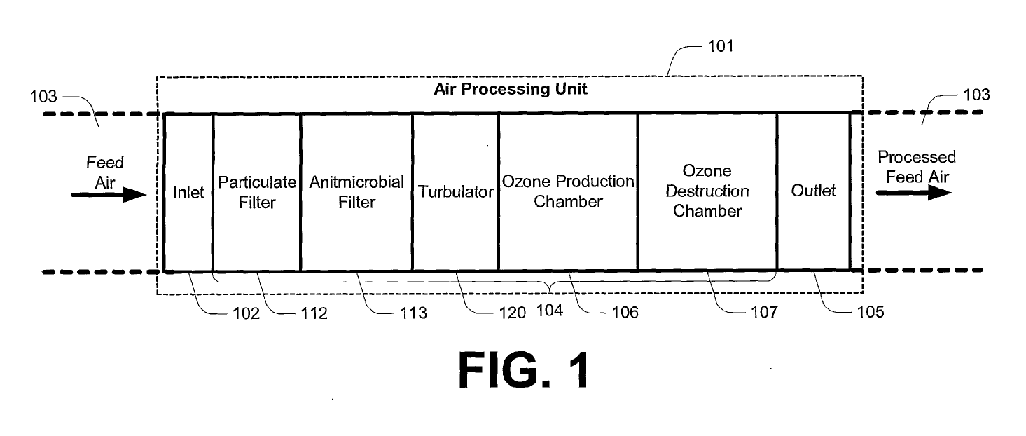

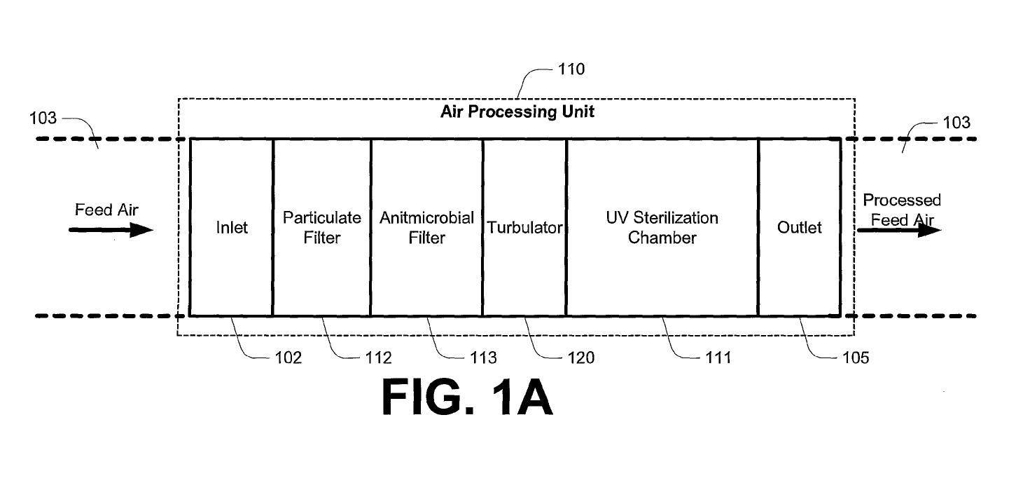

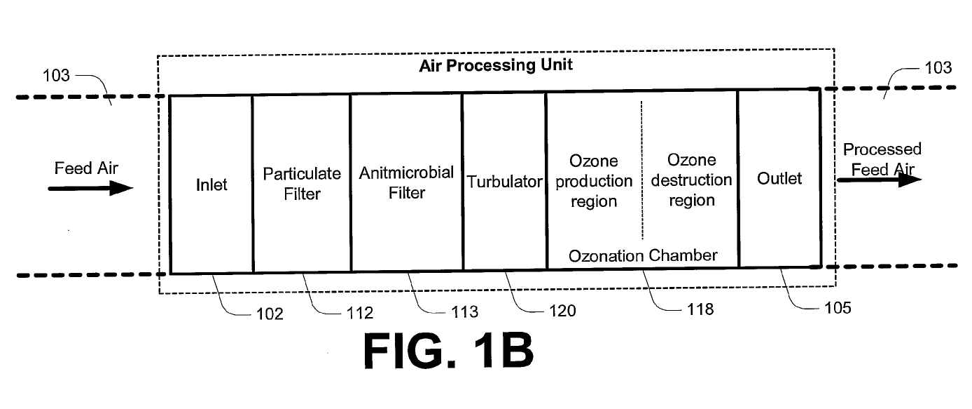

FIG. 1 shows a system for managing air quality according to an embodiment of the present invention, in the form of an air processing unit 101. Unit 101 includes an inlet 102 for receiving feed air from a unidirectional air passage 103. A treatment path 104 is coupled to inlet 102 for receiving, processing and passing the feed air to remove one or more air contaminants. An outlet 105 is coupled to path 104 for passing the feed air to the passage 103 at a location downstream of inlet 102. The pressure drop between the inlet and outlet is less than a predetermined value.

In the present embodiment unit 101 is designed for utilization within an existing air circulation system, for example by insertion into a ventilation duct. Feed air is provided under pressure in a unidirectional fashion though an arrangement of such air ducts supplied by a circulation system from one or more intake locations to one or more redistribution locations. By designing and operating unit 101 in a manner to minimize the pressure drop between inlet 102 and outlet 105 to less than a predetermined value it is possible to correspondingly contain energy overheads associated with the providing the necessary pressures to encourage air flow in the circulation system, often resulting in significant cost savings. Various embodiments of the present invention are directed towards containment of energy overheads whilst providing effective air quality improvement functionalities.

Unit 101 is particularly adapted for the purposes of air sterilization and odor removal. In relation to odor removal, unit 101 includes an ozone production chamber 106 having a first UV light source for emitting UV light at an ozone production wavelength for converting at least some oxygen molecules in the feed air into ozone molecules. This ozone production wavelength is in the present embodiment 185 nanometers, however alternate ozone production wavelengths are used in other embodiments. The UV light in the present embodiment is provided by a plurality of UV light tubes specially adapted to provide the desired wavelength and positioned parallel and substantially equidistant from one another such that the air obtains a relatively constant exposure to ultraviolet radiation. Furthermore, the tubes are provided to be substantially coaxial with path 5. Alternate tube configurations are used on other embodiments, and some examples are provided further below.

UV light provided at the ozone production wavelength breaks down some oxygen molecules into individual oxygen atoms. These oxygen atoms inherently look to stabilize, and in doing so combine with existing oxygen molecules to form ozone molecules. The ozone molecules produced break down odor forming compounds in the air by a process of oxidation. It will be appreciated that this removes odors from the feed air, and the effectiveness is substantially dependant on the degree of ozone production.

In other embodiments alternate components are provided in path 104 for the purpose of ozone production. These include components for inducing electrical fields configured to produce ozone by ionization, and inputs for providing ozone from a source outside of path 104.

An ozone destruction chamber 107 is provided downstream of chamber 106. Chamber 107 includes a second UV light source for emitting UV light at an ozone destruction wavelength for converting at least some—and ideally all—of the ozone molecules into oxygen molecules. In the present embodiment the ozone destruction wavelength is 254 nanometers, however other appropriate wavelengths will be known by those skilled in the art and implemented in other embodiments. In overview, ozone molecules are converted into individual oxygen atoms, which subsequently form into oxygen molecules.

In some embodiments ozone production and ozone destruction are carried out in a common chamber, such as ozonation chamber 118 in FIG. 1B. In some embodiments a single UV tube is used to provide both ozone production and destruction functionalities. In particular, a first portion of tube is coated to produce UV at 185 nanometers and a second portion is coated to produce UV at 254 nanometers.

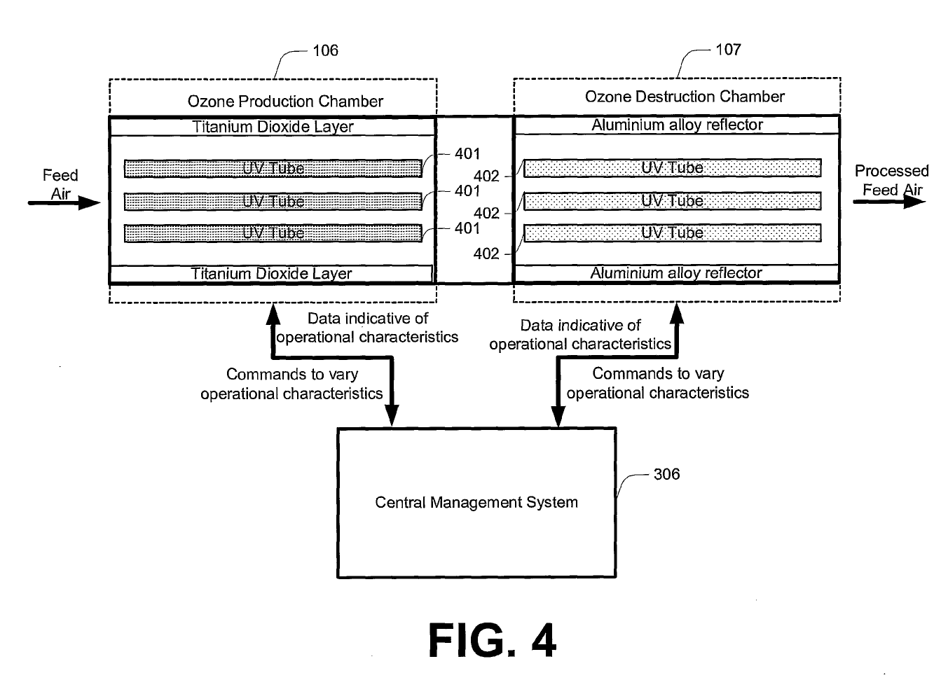

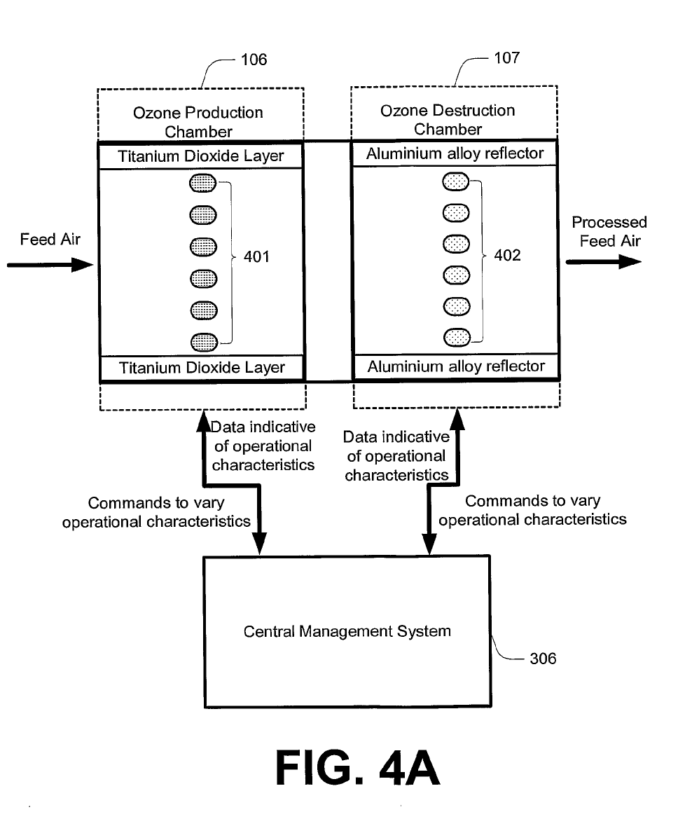

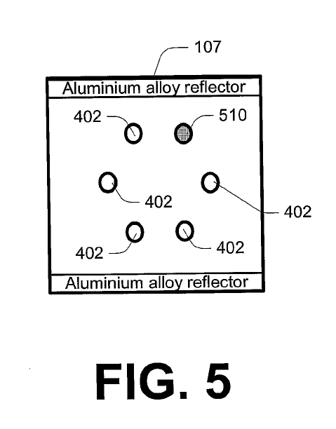

In some embodiments additional steps are taken to improve ozone production and destruction. For example, in the present embodiment a turbulator, in the form of a low resistance fixed blade impellor 120, is provided upstream of chamber 106. In some embodiments additional and/or alternate turbulators are used. For example, in one embodiment an additional impellor is provided intermediate chambers 106 and 107. In another embodiment air turbulation is achieved using fixed air-directing formations for directing airflow into a substantially circular vortex within the chambers. It will be appreciated that the degree of turbulation is balanced with pressure drop considerations. In some embodiments catalysts and/or reflectors are provided in chambers 106 and 107—for example a titanium dioxide coating on surfaces in chamber 106 for encouraging odor removal and/or an aluminum alloy reflector array in chamber 107 for encouraging ozone destruction. In the case of the latter, the reflector arrays effectively increase the dosage of UV radiation within the chamber, resulting in a corresponding in crease in ozone destruction. One particularly useful aluminum alloy is sold under the trade name Alanod. Exemplary ozone production and destruction chambers are illustrated in FIG. 4, FIG. 4A and FIG. 5, which are discussed further below. Throughout the specification, wherever a titanium dioxide layer or surface is used, this is optionally replaced by an alternate reflective layer or surface.

Whilst the UV light sources of unit 101 are in this embodiment optimized for odor removal, in other embodiments UV light sources are optimized for alternate purposes. For example, in the embodiment of FIG. 1A a unit 110 includes a chamber 111 having a UV light source optimized for air sterilization. Although various implementation embodiments of the invention are described by reference to odor reduction units like unit 101 or air sterilization units like unit 110, it will be appreciated that such units are readily interchanged.

It will be appreciated that, although UV light sources of unit 101 are in this embodiment optimized for odor removal, there remains a significant sterilization effect, which results in pathogen removal.

Referring again to FIG. 1, path 104 includes a particulate air filter 112. Filter 112 is a course filter for trapping relatively large particles in the feed air, such as dust or seeds. Filter selection is based on a balance of considerations relating to particle size and backpressure, the underlying notion in this case being to sacrifice particle size in favor of a lower backpressure such that pressure drop is contained. Filter selection is typically based on a threshold particle size, the selected filter trapping particles larger than this threshold size. In some embodiments the threshold size is 10 microns, whilst in the present embodiment the threshold size is the order of 100 microns or larger. In the present embodiment pathogens of under 100 microns are dealt with downstream of the particulate filter.

Filter 112 is provided upstream of an antimicrobial filter 113. An important aspect of this design is to reduce the clogging of a relatively sophisticated antimicrobial filter with large particles. Furthermore, whereas some known particulate filters—typically those with high resistances to airflow that equate to high pressure drops—are designed to trap viruses and other airborne pathogens, in this instance such high grade filters are not required given that viruses and other airborne pathogens are intended to be dealt with downstream by filter 113.

Filter 113 is adapted for killing one or more airborne pathogens, particularly microorganisms. To this end, filter 113 includes one or more of surfaces that are coated with an antimicrobial agent having a pathogen reduction rate of between 50% and 90%, and preferably upwards or 60%, 70%, 80%. In some embodiments more effective agents are used.

The antimicrobial agent of the present embodiment provides something similar to a sharp mesh, which effectively pierces and damages cell membranes of microorganisms as they pass through filter 113. For example, bacteria and other pathogens have a size significantly greater than the size of “gaps” in the sharp mesh. It will be appreciated that the analogy of a sharp mesh is not strictly scientifically accurate, and is provided for the sake of convenient explanation only. Consequently, the filter does not hold any live bacteria within itself. Moreover, the present approach is distinguished from microorganism collection (such as in certain HEPA filters) or the use of biocides (which may be hazardous to human health).

In some embodiments the antimicrobial filter includes as an active ingredient a quaternary ammonium salt. In some embodiments the salt is either a chloride or bromide salt. Preferably the salt includes as a substitute to a nitrogen atom a silane group, which in some cases is a trialkyloxysilane group or trimethyloxysilane group. The nitrogen atom is in some embodiments substituted with the other alkyl groups—at least one or which is preferably methyl and at least one of which is preferable a C₈ to C₂₀ alkyl. In some embodiments the salt has the following general structure:

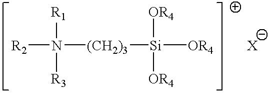

Where:
R₁ is methyl.
R₂ is methyl or optionally C₈ to C₂₀ alkyl.
R₃ is a C₈ to C₂₀ alkyl, in some embodiments tetradecyle or octadecyle.
R₄ is a C₁ to C₄ alkyl, in some embodiments methyl.
X is chlorine or bromine.

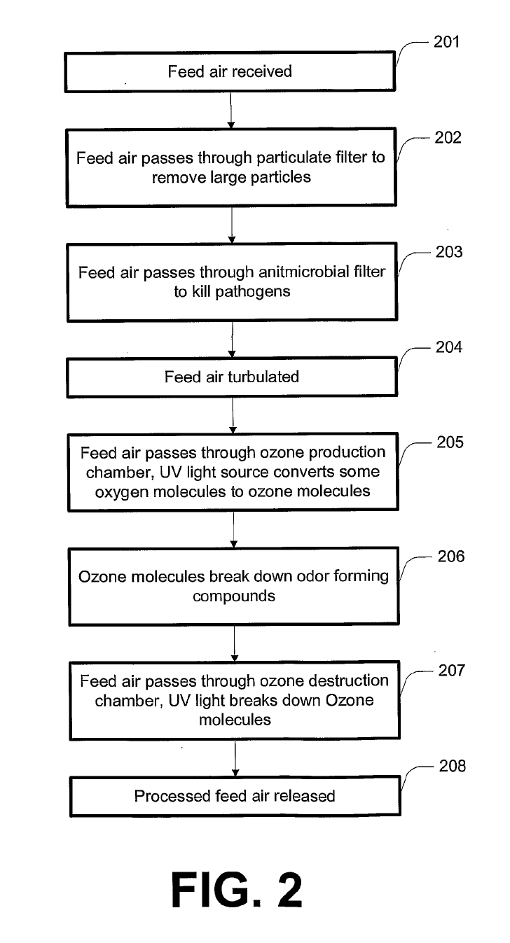

An overview of the operation of unit 101 is provided in FIG. 2. Feed air is received at 201, and this feed air is passed through filter 112 at 202 to remove large particles. The feed air is then passed through filter 113 at 203 to substantially reduce the concentration of pathogens. At 204 the feed is turbulated, and subsequently passed through chamber 106 at 205. At 206 odor is reduced by the operation of ozone, and the ozone is then substantially destroyed in chamber 107 at 207. Additionally, UV light in chambers 106 and 107 removes additional pathogens, increasing the effective pathogen removal rate along path 104. The processed air is released at 208 for recirculation or further downstream processing.

Air processing systems such as units 101 and 110 are effective against substantially all classes of problem microbes, including bacteria, fungi, yeasts and moulds (and their spores), and, viruses. Products utilizing such technology are particularly suitable for use in facilities ranging from hospitals and government embassies to food production lines and real estate developments. Equally importantly, the technology assists contain risks associated with airborne pathogens and threats such as SARS, Avian Influenza, drug-resistant TB, MRSA and bio-terrorism.

As foreshadowed, the pressure drop between inlet 102 and outlet 105 is less than a predetermined value. The rationale for containing the pressure drop relates to corresponding containment of energy overheads in an air circulation system that provides the continuous feed of feed air. In some embodiments the predetermined value is a pressure drop of between 30 Pa to 90 Pa for a flow rate of about 3 linear meters per second. It will be appreciated that pressure drop is non-linearly related to flow rate. More preferably the predetermined value is a pressure drop of between 50 Pa to 75 Pa or between 30 Pa to 75 Pa for a flow rate of about 3 linear meters.

The treatment path in the present embodiment provides a substantially linear pathogen reduction rate across the spectrum of pathogens sized between 0.01 and 100 microns, and particularly 0.01 and 0.03 microns. This reduction rate is based on a comparison of the concentration of pathogens at the inlet and the concentration of pathogens at the outlet. It will be appreciated that pathogen reduction is typically non-linear particularly across the latter range in devices that make use of particulate filtering techniques for pathogen reduction.

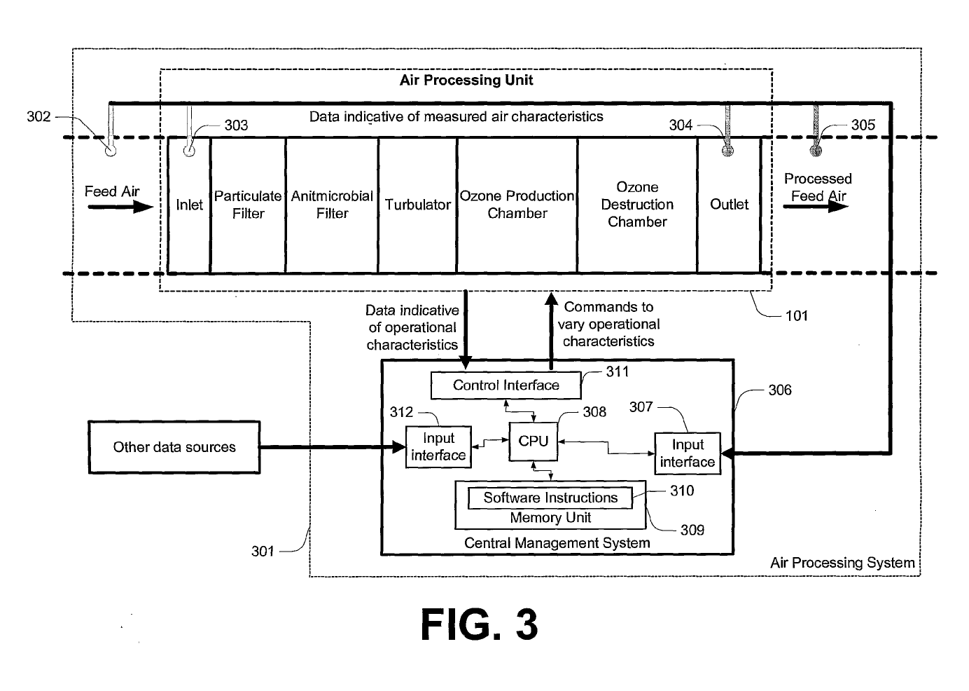

FIG. 3 shows an air processing system 301 including unit 101. System 301 further includes a plurality of sensors 302 to 305. These sensors are each adapted for measuring one or more air characteristics in or adjacent treatment path 104.

Sensors 302 to 305 are illustrated schematically only and described by reference to functionalities to provide an indication of typical locations and uses of such sensors. In practical implementations various known sensors are implemented in place of any of sensors 302 to 305, and in some cases one or more of sensors 302 to 305 are individually defined by a plurality of known sensors. In some embodiments additional similar sensors are provided in either or both of the ozone production and ozone destruction chambers. The sensors in question include sensors for measuring air characteristics such as:
Odor levels. One class of sensors particularly adapted for measuring odor levels are commonly referred to as “e-noses”.
Ozone levels. Some discussion of the rationale for ozone measurement is provided below.
Flow rate. Measuring flow rate at various points in and adjacent path 104 allow for analysis of pressure drop.
Contaminant levels in a board sense, including particulate levels and pathogen levels.

Appropriate sensors for achieving these purposes will be known to and readily implemented by those skilled in the art.

Sensors 302 to 305 are coupled to a central controller in the form of a management system 306. System 306 continuously or periodically receives measurements from sensors 302 to 305 via an input interface 307. A central processing unit CPU 308 operates in conjunction with a memory unit 309 and software instructions 310 for analysis of the measured air characteristics. Software instructions 310 also provide instructions for, among other things, the continued and varied operation of unit 101 and its individual components.

CPU 308 also receives and analyses via a control interface 311 data indicative of operational characteristics of unit 301. These operational characteristics include power consumption, UV emission ratings, UV tube operation, and so on. Additionally, CPU 308 receives via a second input interface 312 data indicative of other factors from other components, which is also analyzed. These other factors typically include flow rates, pressure drops, power consumption and/or contaminant levels associated with other aspects of a wider air recirculation system.

CPU 308 analyses the various data received and in response selectively provides signals to implement subsequent procedures. An important class of signal instructs control interface 308 to vary one or more externally variable operational characteristics of unit 101. Other signals provide information to user interfaces and maintain a log of past activity and a database, such a database optionally being locally or remotely maintained.

In some embodiments instructions 310 are particularly adapted to manage the risk of ozone emission. It will be appreciated that ozone gas is harmful to humans, and as such there is a particular sensitivity to avoid its release into human environments. Provided below are some exemplary techniques implemented by system 301 in various embodiments for managing the risk of ozone emission. It will be appreciated that in other embodiments combinations of these and/or alternate techniques are adopted.
In one embodiment sensor 302 includes an e-nose for measuring the level of odor being drawn into unit 101. CPU 308 is responsive to this measurement for optionally increasing or decreasing ozone production—for example by activating or deactivating one or more ozone production tubes—such that ozone production is controlled to correspond with the level of odor removal required. By reducing ozone production capacity in chamber 106 without adjusting ozone destruction capacity in chamber 107 the chance of ozone clearing chamber 107 without being destroyed is correspondingly reduced.
In one embodiment sensor 304 includes an ozone measurement meter for measuring ozone levels downstream of chamber 107. If the ozone levels exceed a predetermined threshold deemed acceptable for human wellbeing CPU 308 provides a signal instructing unit 101 to decreasing ozone production in chamber 106, or if possible increase ozone destruction in chamber 107. In some cases outlet 105 is closed or similar action taken to prevent the release of ozone into a human environment. In one embodiment a backup UV destruction chamber is provided downstream of the sensor, and invoked by CPU 308 only when ozone levels immediately downstream of chamber 107 exceed the set threshold.
In one embodiment the operational characteristics received at interface 311 include details of the number of operative and/or inoperative UV tubes in chambers 106 and 107. As shown in FIG. 4, chambers 106 and 107 respectively include ozone production UV tubes 401 and ozone destruction UV tubes 402. These tubes inherently deteriorate and fail over time, often in an unpredictable manner. CPU 308 is responsive to the failure of an ozone destruction tube 402 deactivating one or more ozone production tubes or, where possible activating a spare or temporarily and purposively ozone production tube—such as tube 510 shown in FIG. 5. In some embodiments chambers 106 and/or 107 carry one or more backup tubes such that unit 101 is able to operate at full capacity despite the failure of a UV tube. CPU 308 in some embodiments provides one or more system administrators with a message indicative of a reminder or instruction to change one or more failed tubes, in some instances this message being accompanied by a report on unit performance.

It will be appreciated that, as a general principle, reducing the risk of ozone emission involves ensuring that ozone destruction outweighs ozone production. In some embodiments the target ratio of ozone production to ozone destruction is 1:10. That is, the system is configured such that during normal operation there is capacity to destroy ten times the amount of ozone that is produced, thereby reducing the risk of ozone emission into the environment.

In some embodiments instructions 310 are particularly adapted to provide for efficient power consumption by unit 101. For example, the analysis of measurements made by sensors 302 or 303 provides an indication of the extent to which air processing is required, and the power level of components within device 101 are varied to provide an adequate level of processing based on this analysis. For example, if odors measured upstream are found to fall within acceptable levels, the UV sources in chambers 106 and 107 are temporarily deactivated. In one such case the UV source in chamber 107 remains at least partially active for air sterilization purposes.

System 301 not only provides a high effective pathogen reduction rate for a relatively low pressure drop, but also allows ozone dispersion to be contained below threshold levels in compliance with predetermined standards.

FIG. 4A illustrates an alternate UV tube configuration to that of FIG. 4. In the embodiment of FIG. 4A tubes 401 and 402 are provided normal to the airflow path. This alternate configuration is particularly useful given that it allows for relatively shallow units.

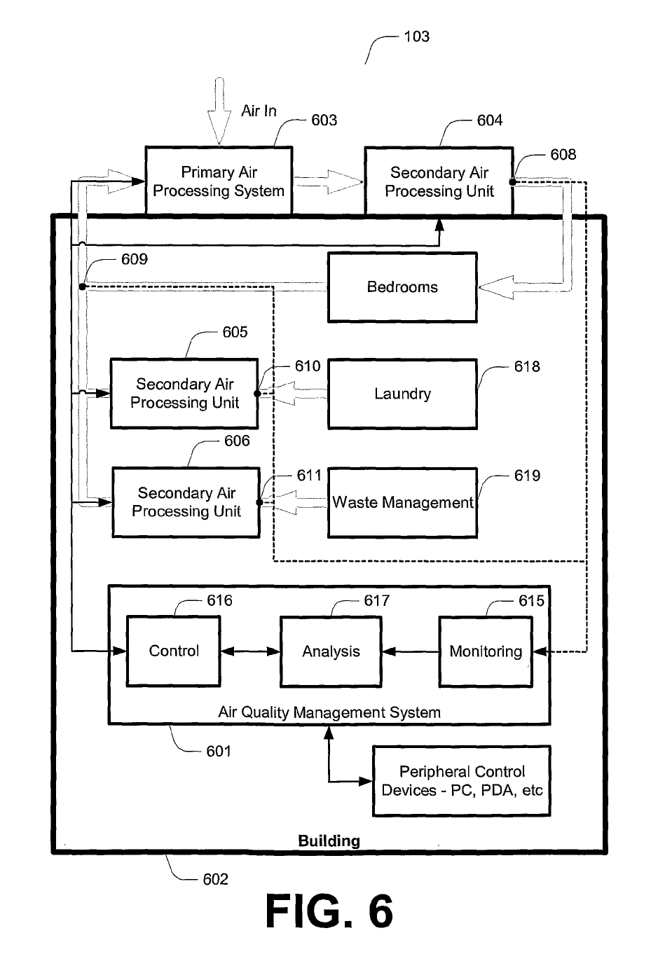

In some embodiments a management system such as system 306 is expanded to provide air quality management functionalities for a multi-zonal environment such as a high-rise building, hospital, seagoing vessel or the like. An example along such lines is provided in FIG. 6, which illustrates an air quality management system 601 for a building 602.

System 601 operates in conjunction with a primary air processing system 603 and a plurality of secondary air processing units 604 to 606. Units 604 to 606 are primarily odor and pathogen removal units, and considered to be substantially the same as unit 101 described above. In some embodiments more or fewer such units are used, and it will be appreciated that the present example is somewhat simplified for the sake of convenient illustration.

In the present embodiment system 601 and units 604 to 606 are installed in a building 602 that is already served by an existing primary air processing system 603. This installation allows for air quality management considerations to be applied with a greater deal of efficiency and a reduced degree of risk.

System 603 is, in absence of system 601 and units 604 to 606, responsible for filtering air for recirculation, and selectively introducing additional air from an external source. It is further responsible for applying power throughout the recirculation pathway to encourage airflow. In the present embodiment system 603 is shown as a rooftop air management system—it will be appreciated that this is for the sake of convenient representation only, and in practice system 603 includes both rooftop components and components spread throughout building 602. For the sake of the present example, it is considered that system 603 includes a rooftop chiller system, a rooftop air intake system, and also an air-recirculation pathway, illustrated schematically as grey arrowed lines in FIG. 6. This pathway is defined collectively by ducts, vents and the like that carry a unidirectional flow of air through building 602 for the purpose of processing and recirculation.

In some embodiments system 603 further includes an NBC (Nuclear, Biological and Chemical) filtration system and/or other air processing systems.

In this example, units 604 to 606 are installed for two major purposes. Firstly, odor removal. This is achieved using an ozone technique as discussed throughout the present disclosure. Secondly, pathogen removal. This is achieved using combination of an antimicrobial filter and UV treatment by ozone production and/or destruction tubes. Particularly relevant to the second purpose, units 604 to 606 reduce the need for energy hungry HEPA filtration units within building 602. It is assumed that system 603 initially included one or more HEPA units, and some or all of these units were either removed or retrofitted with less resistant filters given their redundancy in the presence of units 602 to 604.

In the present embodiment sensor arrays 608 to 611 are provided at locations within the air-recirculation pathway for measuring air characteristics. Similarly to the example of FIG. 4, air characteristic measurements made by these sensors are provided to system 601 for analysis. Also provided to system 601 to assist in such analysis are operational characteristics for system 603 and units 604 to 606. Data indicative of air characteristic measurements are provided to a monitoring interface 615, whilst data indicative of operational characteristics are provided to a control interface 616. Control interface 616, similarly to interface 311, is adapted not only to receive data indicative of operational characteristics, but also to adjust these operational characteristics where possible. Data from other sources is also received and analyzed, such as building power consumption and occupancy rates.

Analysis within system 601 is preformed by an analysis subsystem 617, which includes an information system having at least one processor and associated memory unit, the memory unit carrying software instructions for allowing implementation by system 601 of various methods described herein. Pursuant to the analysis, subsystem 617 provides instructions to vary operational characteristics of system 603 and units 604 to 606, as well as providing information to peripheral control devices such as networked personal computers or portable devices including PDAs that wirelessly communicate with subsystem 617. Subsystem 617 also provides a user interface to such devices to allow optional manual control of operational characteristics of system 603 and units 604 to 606.

In the present embodiment secondary air processing units are provided at two classes of locations:
Downstream of the rooftop HEPA/intake system. Unit 604 is provided at that location for processing air discharged by the rooftop system and/or air draws from the outside. In the case of the former, contaminants introduced by system 603 (which are particularly common where system 603 includes a rooftop chiller) are removed, as are contaminants introduced by new externally sourced air. Unit 604 reduces the risk of such contaminants being recalculated throughout building 602.
Downstream of “hotspots”. Hotspots are locations in building 602 that are specifically identified to produce a relatively large amount of contaminants. Two examples are provided, being a laundry 618 and a waste management facility 619. Odors and pathogens stemming from those locations are respectively removed form the building's recirculation flow by units 605 and 606 respectively. The primary rationale for providing such units at hotspots is to reduce the strain on similar units downstream, allow for power conservation by selectively disabling units when certain facilities are not in use, reducing the buildup of pathogens in ventilation ducts, and reducing the level of pathogens introduced to the rooftop HEPA system.

There are significant benefits resulting from the provision of a unit such as unit 101 upstream of an existing HEPA filtration system. These benefits include:
Health hazard reduction. HEPA filters are often designed to trap pathogens, and these trapped pathogens often present a serious health risk to persons performing maintenance on the HEPA system—particularly during filter cleaning or replacement. An upstream processing unit such as unit 101 vastly reduces the extent to which viral pathogens are provided to the HEPA system. There is a risk that distinct viruses trapped in HEPA filters could combine and/or mutate to produce more dangerous strains, increasing the risk of pandemics.
Optional power savings. Given that pathogen reduction is performed in a unit distinct of the HEPA unit, a lower grade HEPA filter may be installed in an existing HEPA system to reduce the pressure drop across that system and thereby reduce energy consumption. Indeed, in some cases the use of a unit 101 renders an existing HEPA system unnecessary, and as such the HEPA system may be removed. This is particularly the case where a HEPA system is implemented for the purpose of pathogen removal—the use of a unit is able to provide similar pathogen removal effects at only a portion of the energy consumption overhead.

Embodiments of the present invention include methods for installing units such as unit 101 to improve the efficiency of or eliminate the need for HEPA filters, thereby to improve the efficiency of recirculation systems on the whole. For example, the efficiency of a recirculation system is increased by enabling a higher than usual air recirculation ratio, whilst maintaining air quality by eliminating odors and pathogens. Those skilled in the art will recognize such methods from the above disclosure

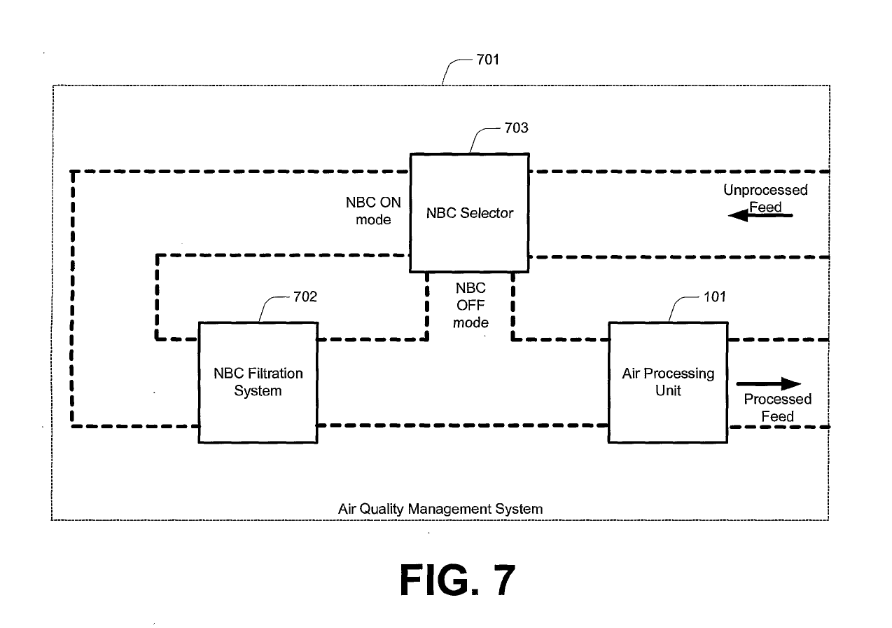

FIG. 7 illustrates an embodiment in the form of an air quality management system 701. System 701 includes unit 101 (or a variation thereon) in conjunction with an NBC filtration system 702. The underlying rationale behind this embodiment is that an NBC filtration system is energy consuming to operate. To save energy a NBC selector unit 703 is installed, which typically includes one or a series of selector valves installed in an air recirculation system at one or more locations upstream of system 703. Selector unit 703 is operable in as NBC ON position in which air is directed through the NBC system and then on to unit 101, and a, NBC OFF position in which air is directed immediately towards unit 101. A control unit (not shown) is responsible for the coordination of selector unit 703, NBC system 702 and unit 101. In particular, NBC ON position is selected only when the NBC system is activated. In some embodiments the control unit is also responsive to the activation of the NBC unit for adjusting air flow rates in the overall circulation system to account for an increased pressure drop brought about by operation of the NBC system. In some embodiments unit 101 is operated under different operational characteristics depending on whether the NBC system is in use.

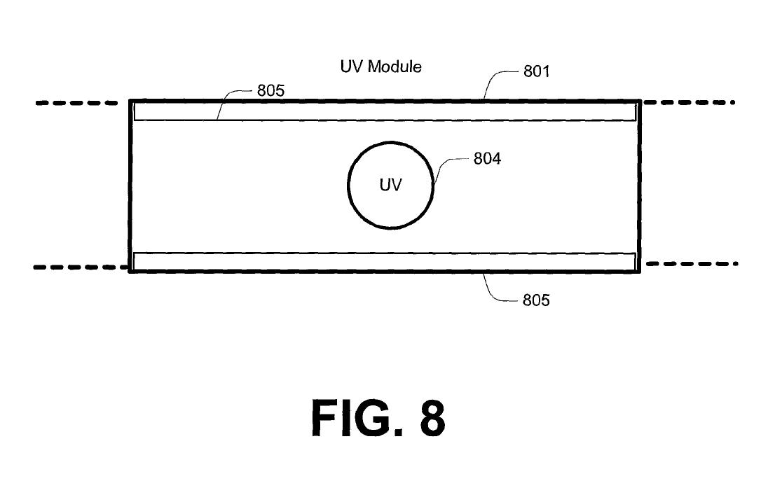

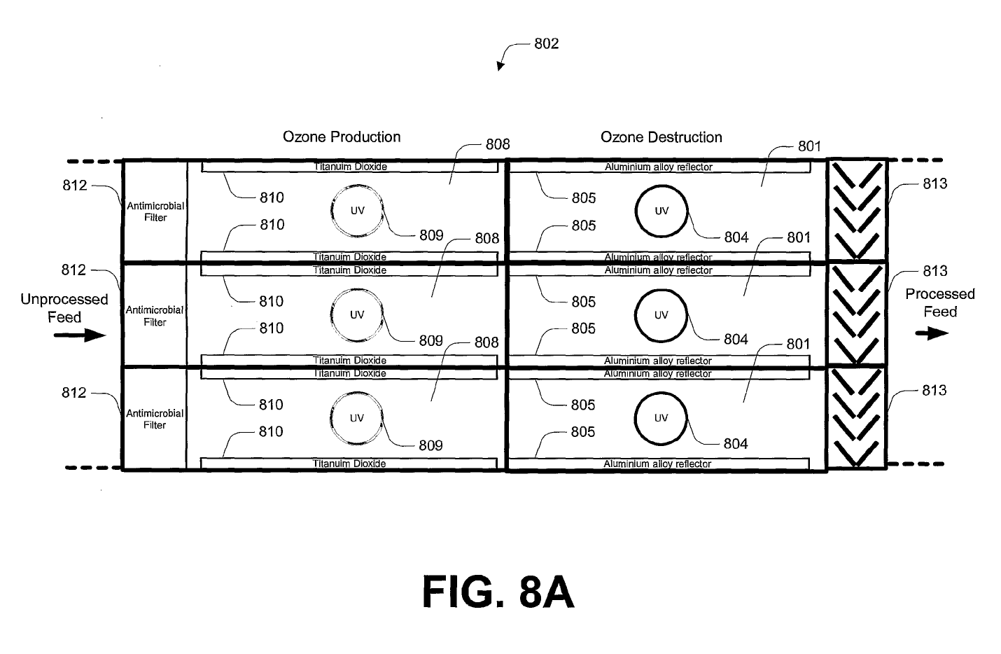

FIG. 8 to FIG. 13A illustrate embodiments of the invention making use of modular designs. The underlying design principle between these embodiments is to provide modules that in combination provide air processing systems, the individual modules being stacked or otherwise combined in parallel and/or series to fill an air inlet/outlet of given dimensions. That is, a frame is provided for insertion in a duct or other location, this frame being configured to removably house a plurality of modules. Where appropriate, the frame provides terminals that, upon the mounting of a module to the frame, come into electrical contact with complementary terminals on that module thereby to provide power to the mounted module and allow operation of components such as UV tubes.

The use of a modular design allows components to be conveniently replaced, typically by removal and replacement of a module containing components for replacement. Modules are typically replaced after a given period or after reaching an identifiable state of disrepair. In some instances replaced modules are discarded, in other cases replaced modules are re-fitted with new consumables—such as UV tubes or filters—and subsequently refitted as replacements for another module. The use of modules saves time during installation and maintenance, and further assist in reducing the range of spare parts that need be taken on-site for unit servicing. Furthermore, re-fitting may occur at a central servicing facility. Modular replacement also allows for risk management based on a Mean Time Between Failures type management protocol whereby modules are periodically replaced on a rotation to manage the risk of serious system failures.

FIG. 8 illustrates a UV module 801 for use in a modular air processing system 802. Module 801 includes a rectangular prismatic modular body 803. Module 801 is an ozone destruction module, and includes a UV tube 804 in body 803 for emitting UV light at an ozone destruction wavelength, and Alanod layers 805. Tube 804 is provided normal to the direction of airflow, thereby reducing the overall length of system 802.

In some embodiments a plurality of vertically spaced tubes 804 are used.

FIG. 8A shows a plurality of modules 801 in parallel. Each module 801 has upstream and in series with it a similar module—in the form of an ozone production module 808. A module 808 differs from module 801 in that it includes an ozone production tube 809 and titanium dioxide layers 810. It will be recognized that modules 808 are responsible for ozone production, and modules 801 responsible for ozone destruction.

System 802 includes a plurality of parallel antimicrobial filter modules 812, one such module being provided upstream and in series with each module 808. Each module 812 includes an antimicrobial filter as described above for pathogen removal. In some embodiments these modules are discrete boxes containing filters, whereas in other embodiments these modules are merely filters insertable into guides formed in, on or through module 804. In some embodiments a modules 812 include a particulate filter upstream of the antimicrobial filter. In some embodiments a filter, louver arrangement, reflector array or the like is used to prevent the passage of UV radiation between ozone production and destruction zones. For example, in one embodiment a filter is provided intermediate the ozone production chamber and ozone destruction chamber to prevent or inhibit passage of UV at 185 nanometers in the downstream direction. Similar features are optionally included in other embodiments, such as those discussed below.

Although the examples in FIG. 8A to 12A show a plurality of parallel modules 812, in some embodiments a single module 812 is provided spanning across the upstream ends of a plurality of modules 808.

A plurality of parallel louver modules 813 are provided downstream of modules 801 for preventing the emission of UV outside of system 802. These modules each include an array of louvers having matte black or other absorbent coatings. The underlying rationale is to prevent UV radiation be transmitted externally of system 802, however in some embodiments this concern is outweighed by pressure drop considerations, and louver modules correspondingly omitted.

Although the examples in FIG. 8A to 12A show a plurality of parallel modules 813, in some embodiments a single module 813 is provided spanning across the downstream ends of a plurality of modules 808.

Although not shown in the present illustrations, in some embodiments a structural frame is provided for supporting individual modules for allowing convenient installation and removal. For example, in one embodiment modules are snap-lockingly engagable with a frame.

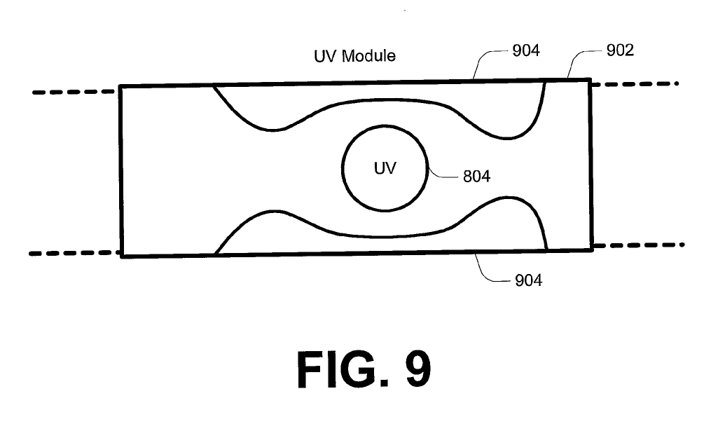

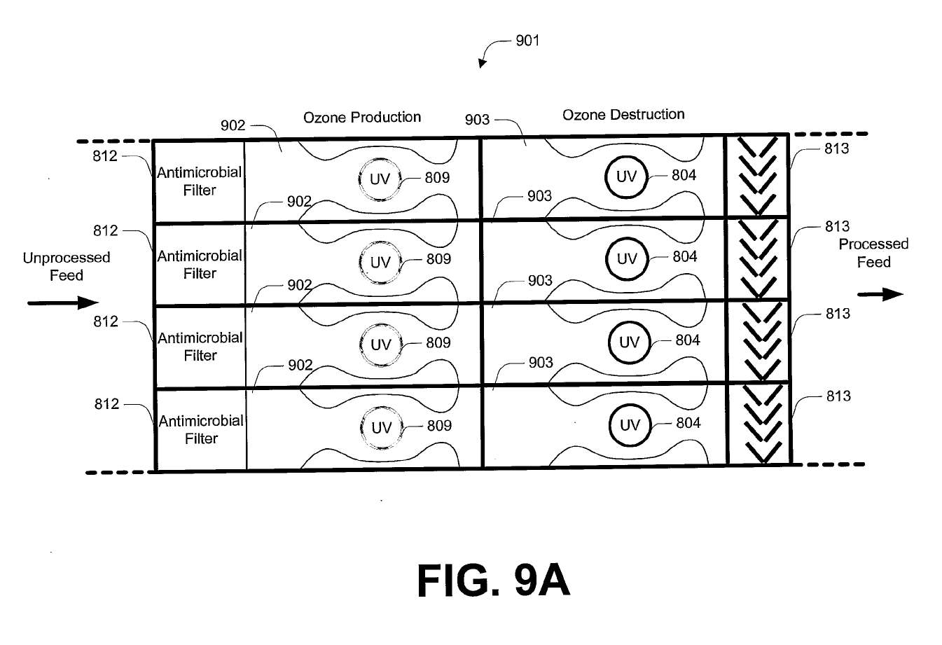

FIGS. 9 and 9A show a similar example to that of FIGS. 8 and 8A, in the form of a modular air processing system 901 including ozone production modules 902 and ozone destruction modules 903. Like components follow numbering conventions of FIG. 8 and FIG. 8A.

The major difference between system 802 and system 901 is the inclusion of fixed formations 904 in the modules. These formations are provided on the interior of the modules for to increase ozone production or destruction capacity, primarily be increasing reflective properties, but also to a degree to encourage turbulence. Typically formations 904 are coated with either titanium dioxide or Alanod to assist respectively either odor removal or ozone destruction. It will be appreciated that production/destruction capacity gains come at the expense of pressure drop minimization.

Formations 904, and similar formations, provide an increase in ozone production in a number of ways. To some extent, they provide for increased air turbulation, which increases UV exposure. To some extent, they guide air to flow in close proximity to UV tubes, thereby increasing UV exposure, which increases with proximity. However, the primary purpose in this embodiment is to provide varying reflective surfaces for increasing UV concentrations at regions within the module.

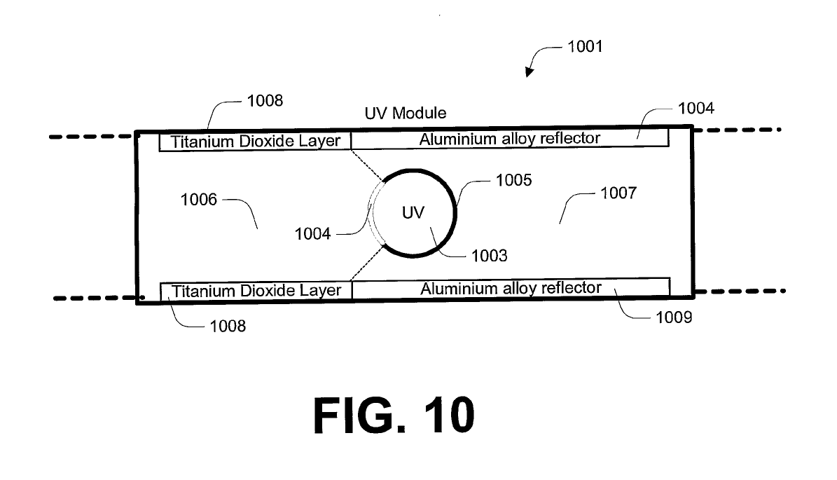

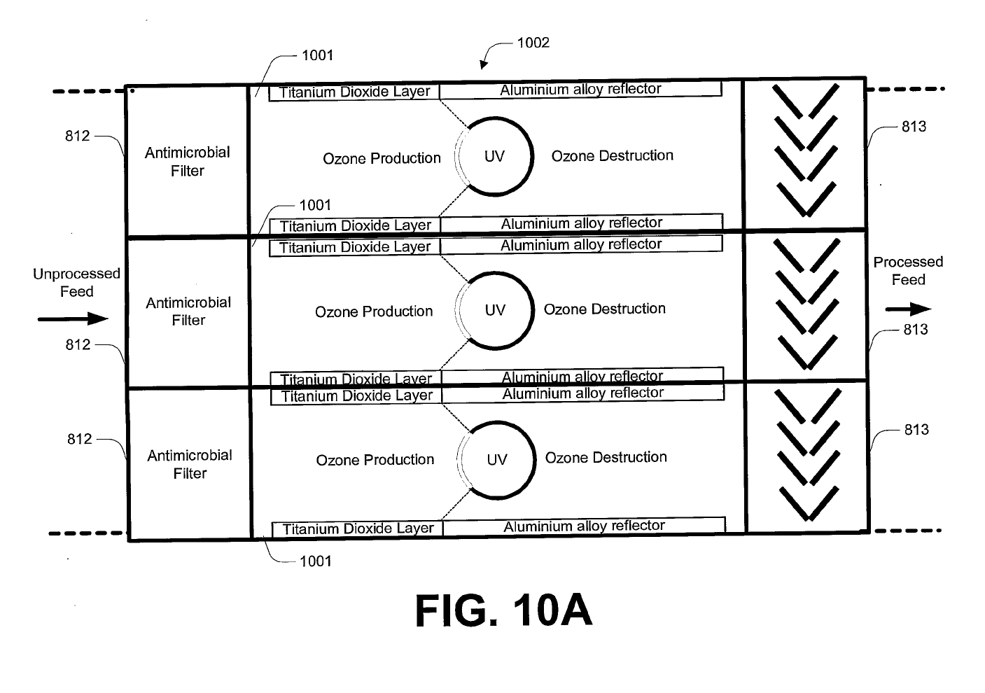

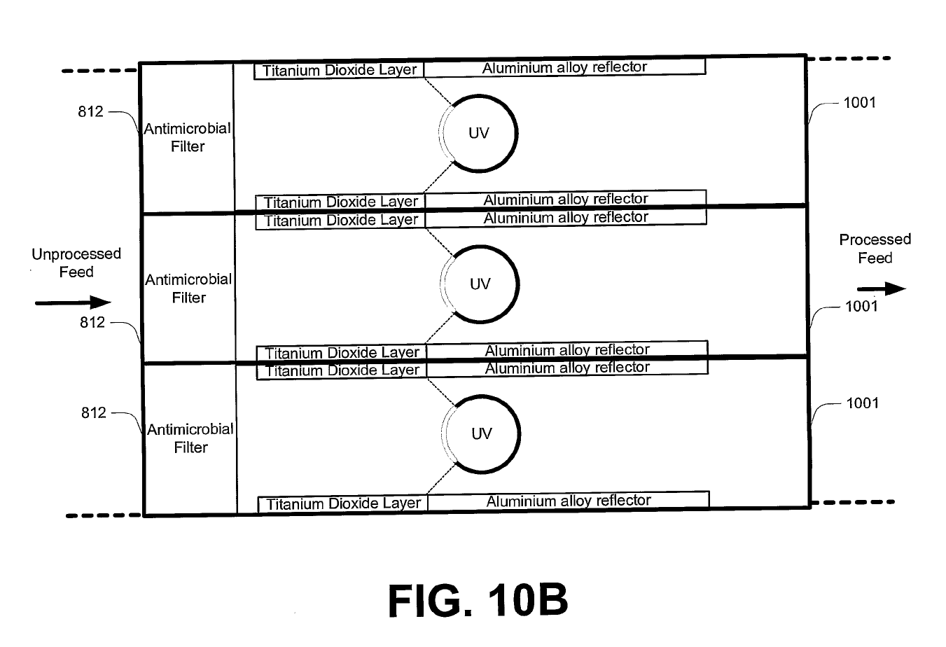

FIG. 10 and FIG. 10A illustrate a module 1001 and modular system 1002. Again, like components follow numbering conventions of previous examples.

Module 1001 is similar to module 801, however it includes a dual purpose UV tube 1003. Tube 1003 includes a first circumferential portion 1004 coated to emit light at an ozone production wavelength and a second circumferential portion 1005 coated to emit light at an ozone destruction wavelength. As such, ozone is produced in an upstream region 1006 and subsequently destroyed in a downstream region 1007. It will be appreciated that portion 1005 is relatively larger to sway the production/destruction balance significantly in favor of destruction.

In the present embodiment modules 1001 include titanium dioxide layers 1008 in region 1006 and Alanod layers 1009 in region 1007. In some embodiments these are not used, and in some embodiments Alanod is used in both regions 1006 and 1007.

The module of FIG. 10 provides for a particularly low pressure drop and shallow physical profile. In the embodiment of FIG. 10B additional advantage of these characteristics is taken by the omission of louver modules 813.

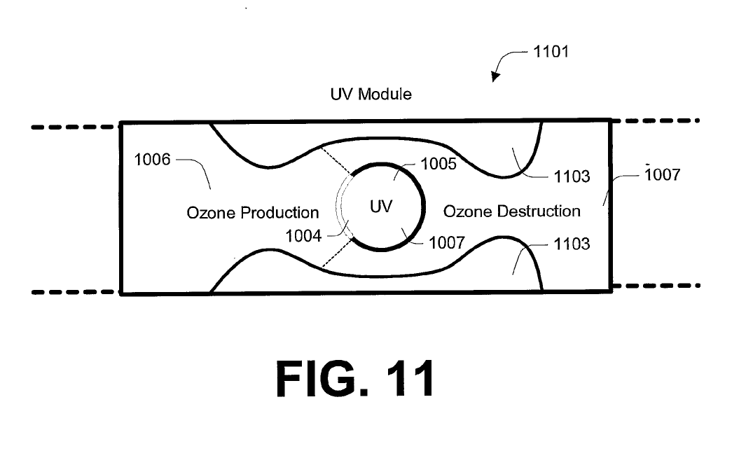

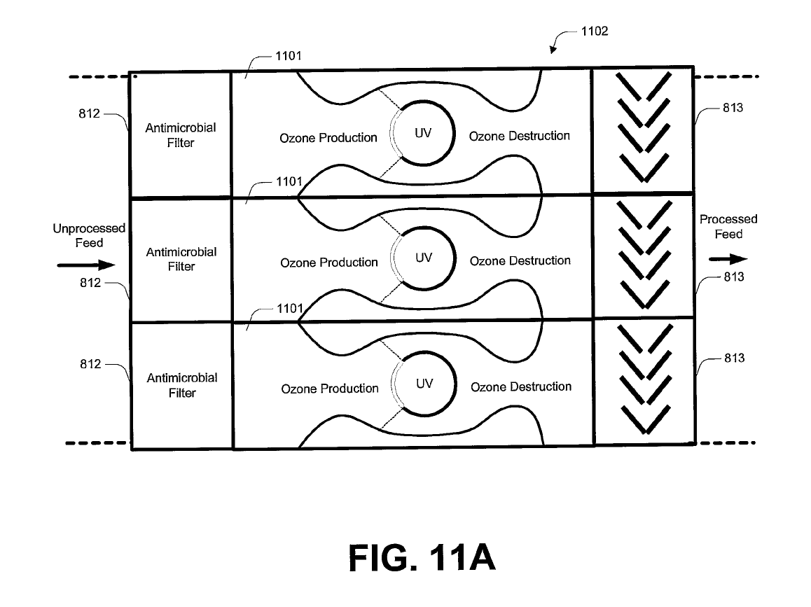

FIG. 11 and FIG. 11A illustrate a module 1101 and modular system 1102. Again, like components follow numbering conventions of previous examples. The primary point of difference between module 1101 and module 1011 is the inclusion of formations 1103, similar to those used in FIGS. 9 and 9A. In the present example each formation typically includes a titanium dioxide coatings in region 1006 and Alanod coatings in region 1007.

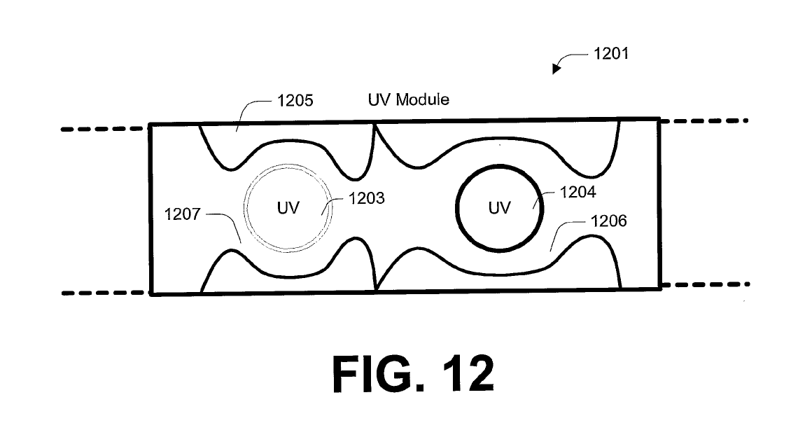

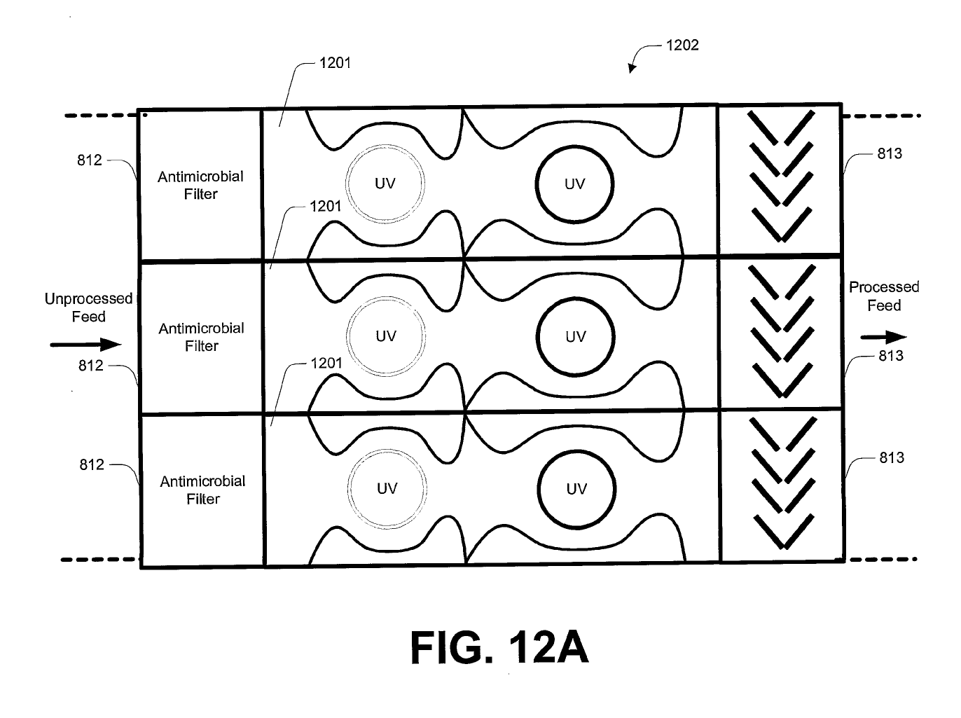

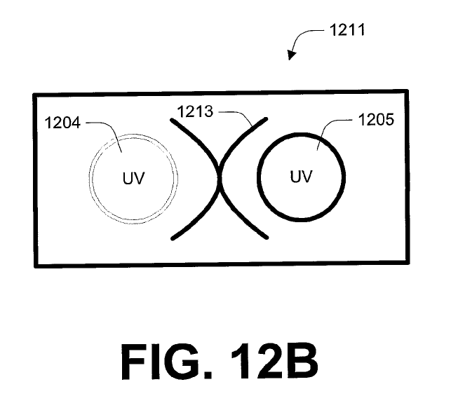

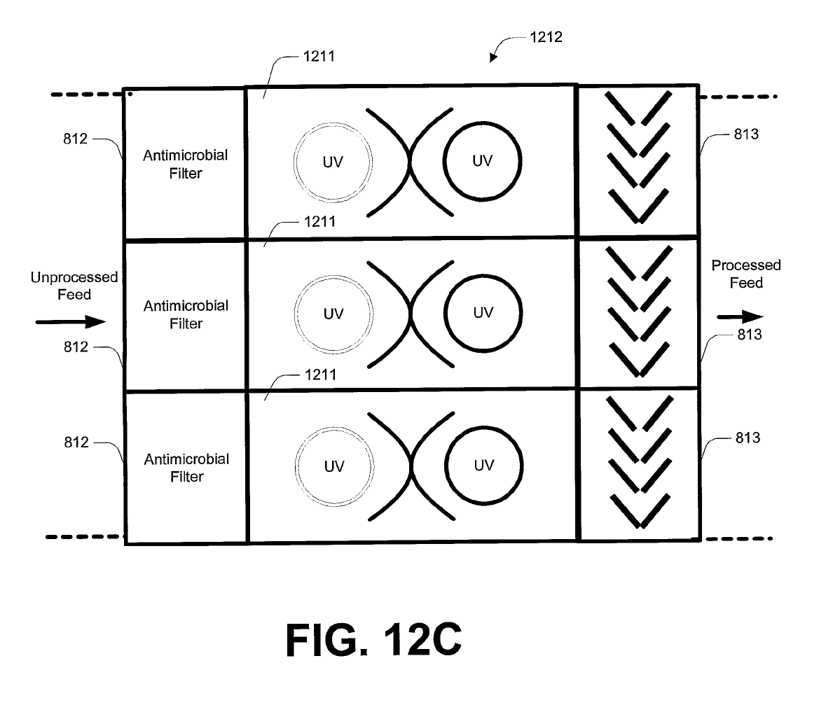

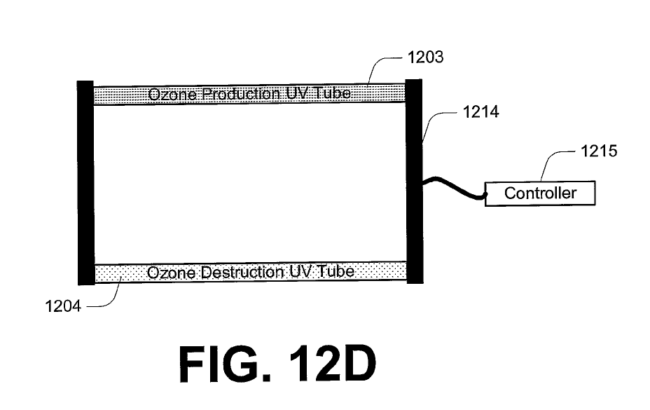

FIG. 12 and FIG. 12A illustrate a module 1201 and modular system 1202. Again, like components follow numbering conventions of previous examples.

Module 1201 includes substantially parallel tubes: an ozone production tube 1203 and an ozone destruction tube 1204. A formation 1205 provides turbulation and provides some UV blocking. In this example ozone is produced in a region 1207 and destroyed in a region 1206—however it will be appreciated that these regions overlap intermediate the tubes. Again, filters, louvers or the like are used in some embodiments to separate ozone production regions from ozone destruction regions. The tubes are configured to provide a relatively higher ozone destruction capacity than production capacity. To this end, tubes 1204 are typically three to six times more powerful than tubes 1203.

FIG. 12B and FIG. 12C illustrate a module 1211 and modular system 1212. Again, like components follow numbering conventions of previous examples.

Module 1211 includes a central reflective member 1213 for functionally separating UV production and destruction regions, whilst increasing UV concentrations in each of these regions. In this embodiment the UV production tube and UV destruction tube are coupled by a frame 1214, as should in FIG. 12D. This frame is coupled to a controller 1215, which monitors the performance of the two tubes. Controller 1215 is responsive to failure of tube 1214 for deactivating tube 1203.

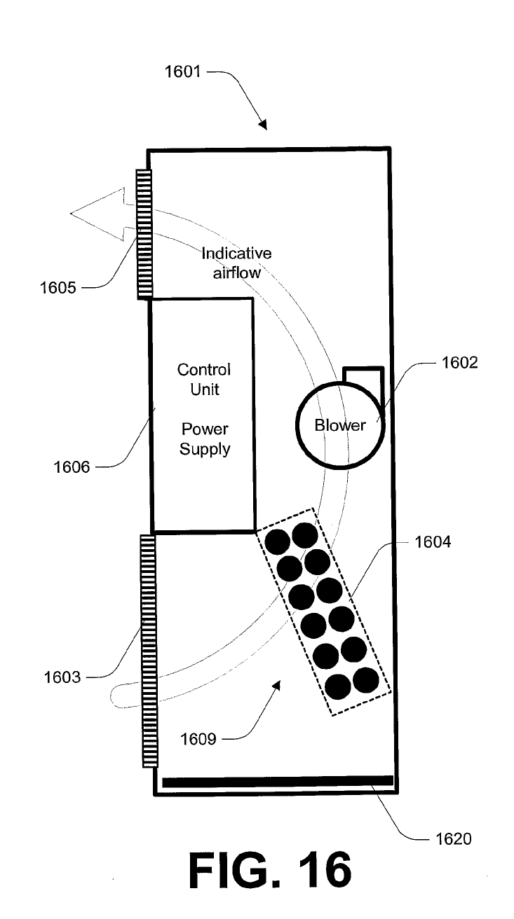

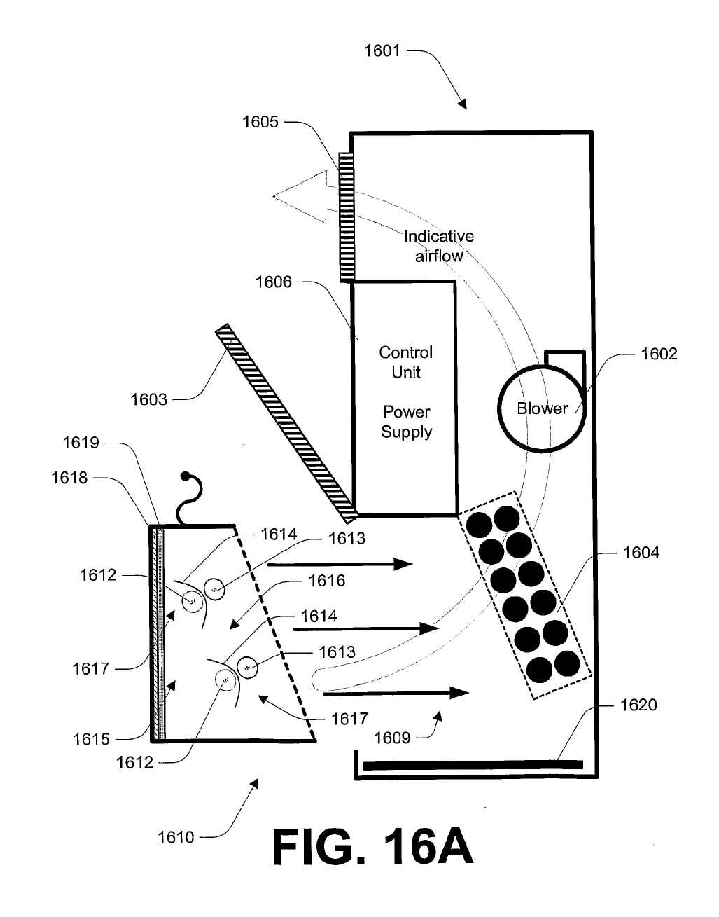

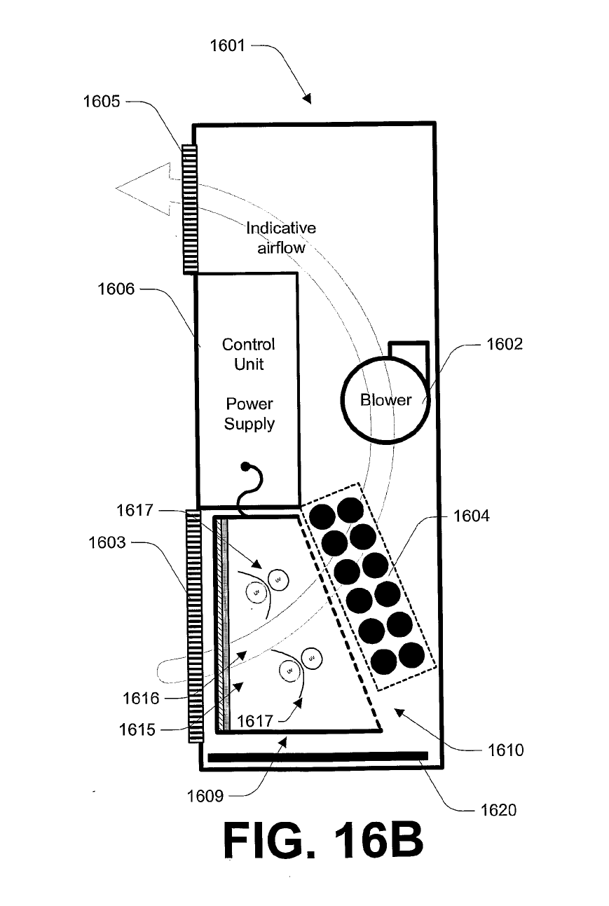

The reflector shown in FIGS. 12A, 12B and 12C includes a portion curved about the ozone production tube, and a portion curved about the ozone destruction tube. In some embodiments with a reflector is used that curves about only one of those tubes, typically the zone production tube. An example of such a reflector is shown in FIG. 16A.

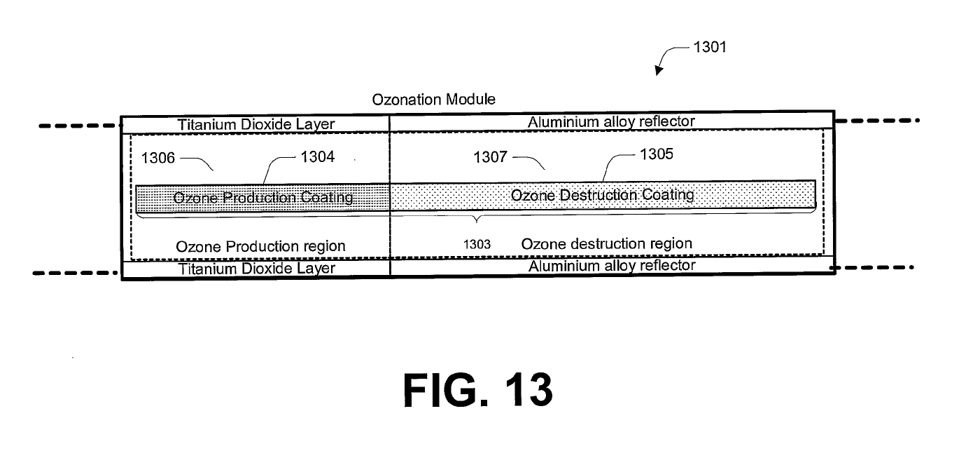

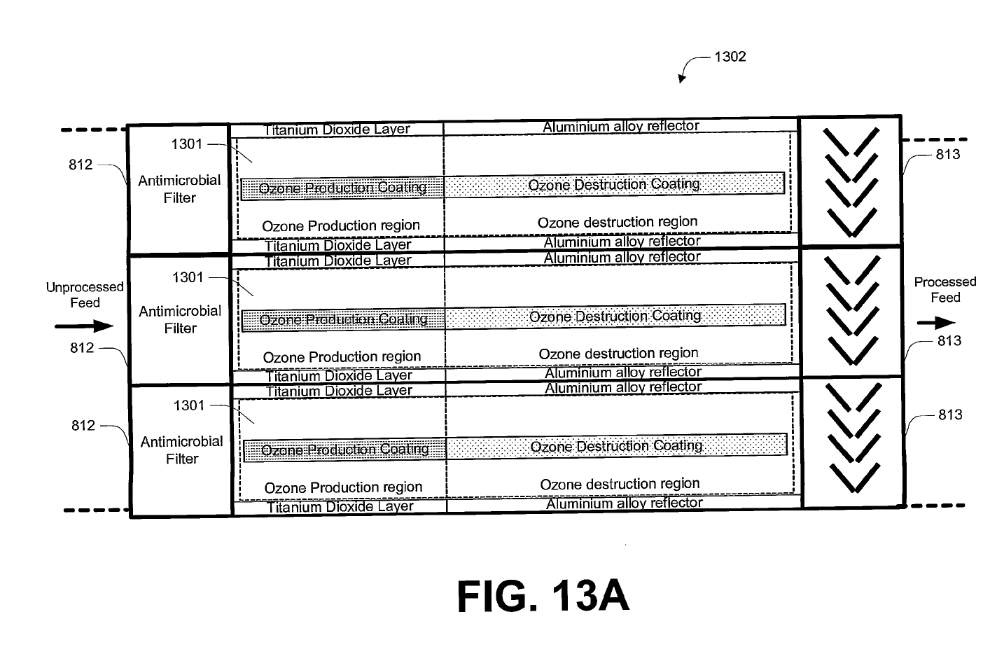

FIG. 13 and FIG. 13A illustrate a module 1301 and modular system 1302. Again, like components follow numbering conventions of previous examples.

Module 1301 includes a plurality of parallel UV tubes 1303, although in the present view only one is shown. In some embodiments modules 1301 only include a singe tube 1303.

Tube 1303 includes a first longitudinal portion 1304 coated to emit light at an ozone production wavelength and a second longitudinal portion 1305 coated to emit light at an ozone destruction wavelength. As such, ozone is produced in an upstream region 1306 and subsequently destroyed in a downstream region 1307. It will be appreciated that portion 1305 is relatively longer to sway the production/destruction balance significantly in favor of destruction.

It will be appreciated that the modular systems described above are readily interchangeable with other air processing units discussed above, for example in the context of system 601.

Modular systems, such as those described above, are in some embodiments used in conjunction with a management system like system 306 described above. The management system received information from sensors and from individual modules, allowing for the monitoring of performance. In some embodiments where a module is found to have failed then some or all modules in series with that module are disabled. For example, if an ozone destruction module 801 fails, then the upstream series ozone destruction module 808 is immediately disabled.

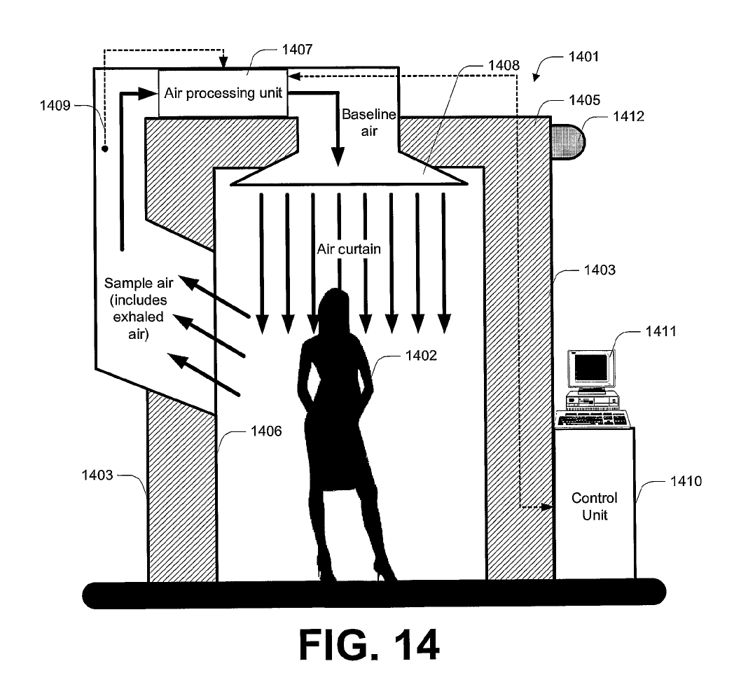

Some embodiments of the present invention are directed to the effective isolation of persons in a social situation. For example, FIG. 14 shows a booth 1401 for analyzing air exhaled by a person 1402.

Booth 1401 includes upwardly sidewalls 1403 supporting an outwardly extending roof 1404. The space between the sidewalls and the roof defines a target zone 1405 in which person 1402 stands.

The underlying structural design rationale is to provide an isolatable environment. Whilst in the present embodiment such an environment is created by two sidewalls, in other embodiments a sealed booth having three or more fully sidewalls connected is instead used. It will be appreciated that single or virtual sidewall embodiments also exists. One relevant consideration is human perception—the more open a booth, the less intimidating it is likely to be found.

An inlet 1406 is formed in one of sidewalls 1403 for substantially outwardly drawing sample air from target zone 1405. An air processing unit 1407—in some embodiments being an air processing unit or modular air processing system as described above—is coupled to the inlet for processing the sample air to produce baseline air having predefined characteristics. In overview, the baseline air is the same as the sample air, however with the vast majority of pathogens removed. An outlet 1408 is formed in the roof 1404 and coupled to unit 1407 for substantially downwardly providing the baseline air to target zone 1405 as an air curtain. Booth 1601 includes a fan or other air circulation means (not shown) for providing the airflow.

Inlet 1406 is formed at or about head height—typically 1 meter and 2.5 meters above the ground. The rationale is to increase the probability of withdrawing air exhaled by person 1402. In general terms, the air flow configuration within booth 1401 is such that outlet 1408 provides baseline air having known characteristics (in some instances measured by an additional sensor adjacent outlet 1408) and inlet 1406 withdraws sample air consisting of baseline air and air exhaled or otherwise affected by person 1402.

A sensor 1409 is provided upstream of unit 1407 to measure pathogen levels in the sample air. This sensor is coupled to unit 1407, which in turn is coupled to a control unit 1410. Control unit 1410 provides an interface via PC 1411 by which an administrator is able to review measured air characteristics and otherwise control unit 1407. The general notion is that differences in characteristics between the sample air and baseline air are caused by person 1402. If pathogens not present in the baseline air are found in the sample air, then control unit 1410 deduces that person 1402 is responsible for these pathogens. If the pathogens are considered to present a risk based on information maintained by control unit 1410 then an alarm 1412 is raised to alert an administrator.

Booth 1401 is in some embodiments configured such that the air within the booth is kept at a lower pressure to the external environment to prevent air escape. Ideally all in the booth is over time treated via unit 1407 and recycled within the booth. Alternately or in combination all air leaving the booth is similarly treated. In some embodiments additional external air is drawn in upstream of unit 1407 but downstream of sensor 1409.

Booth 1401 is conveniently implemented for the purpose of disease management and pandemic risk reduction. It allows for individual persons (or groups of persons in larger embodiments) to be quickly and conveniently analyzed for the presence of contagious airborne infections.

In one implementation booth 1401 is implemented in an airport disembarkation gate. Persons arriving at the airport and using this gate to leave an aircraft are each directed to stand in a booth 1401 for a short period of time—typically less than one minute. Persons found to be exhaling pathogens are identified and, where appropriate, taken into isolation prior to their entry into the general population at their destination.

In a similar implementation alternate scanning techniques are used for pre-screening persons, and only a selection of persons are directed to enter a booth 1401. For example, known thermal imaging techniques are used to assess body temperatures of individual persons in a relatively large group, and a person found to have body temperatures indicative of disease are directed towards a booth 1401 for further analysis.

In one implementation a person's history is taken into account. For example, persons arriving on flights originating from regions of the world known to have higher disease risks—or persons known to have visited such regions in a recent period of time—are identified and directed towards a booth 1401. Such an approach reduces the risk of regional diseases spreading, such as avian flu.

In some embodiments the general body of booth 1401 is structurally defined by an airplane cabin or disembarkation bridge for sampling a large group of persons to determine whether a carrier of a contagious disease is present. It will be appreciated that the general technique of sample air withdrawal, testing, processing by a device herein disclosed, and redistribution is applicable in a variety of other situations.

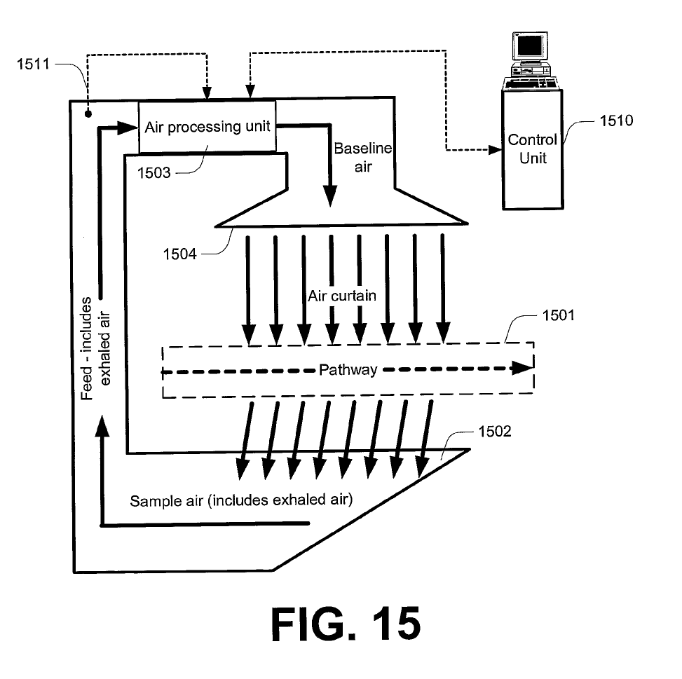

The embodiment of FIG. 15 extends the general concept of booth 1401 to a more open location respiratory isolation context. In the embodiment of FIG. 15 a number of persons follow a common pathway 1501 in a common direction of travel—such as along a hallway, or in a queue. Sample air is withdrawn via an inlet 1502, processed via a unit 1503 to produce baseline air, and the baseline air is provided via an outlet 1505 as an air curtain. The air curtain is provided relative to the inlet to provide a flow of Vaseline air across pathway 1501 such that person on that pathway breath substantially only baseline air, and substantially all exhaled air is withdrawn and processed prior to inhalation by other persons. In this way a number of persons in a social environment are less likely to contaminate one another by way of airborne diseases. In some embodiments the air curtain is provided either downwardly from a ceiling and the inlet is provided on the wall or opposed floor. In some embodiments the air curtain is provided outwardly from a wall and the inlet is provided on an opposed wall or adjacent floor or ceiling. Various configurations are implemented in various embodiments with the underlying intention being to create a flow of air across a plurality of persons and in doing so to reduce or ideally eliminate inhalation air by a first person where that air has been exhaled by a second person and not yet treated.

A control unit 1510 in combination with a sensor 1511 monitor exhaled pathogen levels, and administrators optionally take escalated actions is pathogen levels reach predetermined concern levels.

Embodiments such as that of FIG. 15 are particularly useful in airports, for example in immigration halls where a number of persons from varying points of origin accumulate in queues.

FIG. 16 illustrates a vertical furred-in stack type fan coil air conditioning unit 1601. Unit 1601 includes a circulation module in the form of a blower 1602 for creating a pressure drop thereby to draw air through the unit. Specifically, air is in the first instance drawn from an indoor environment through an inlet vent 1603, which in practical terms is a removable mesh grating covering an aperture. The drawn air then passed through a cooling zone 1604 including an array of cooling coils which serve to cool (or in some cases heat) the air. These coils are managed by a cooling system—typically including a compressor—which is not shown. The air, having past through the cooling zone and as such having been cooled, is then returned to the indoor environment through an outlet vent 1605. In broad terms, air at room temperature is drawn in, cooled, and returned to the room from which it was drawn.

Unit 1601 in this embodiment includes a central logic unit 1606. Logic unit 1606 manages characteristics of airflow rate (by management of the performance of the blower) and cooling characteristics (by managing the temperature of the cooling zone). Unit 1601 also includes, in this embodiment, a performance monitoring interface and a power supply to which separate devices are able to connect.

Unit 1610 is generally indicative of known such units, such as those manufactured and sold by Carrier Corporation as the following models: 42SG Furred-In Stack, 42SH Cabinet, and 42SJ Back-to-Back Furred-In Stack. Embodiments of the invention are configured for specific mounting in and utilization with these models.

FIGS. 16A and 16B show a device and method for managing air quality in accordance with further aspects of the present invention, the device taking the form of device 1610. Device 1610 has a wedge shaped body that is geometrically adapted for insertion into unit 1601. Specifically, unit 1601 includes a void 1609 intermediate vent 1603 and zone 1604, and device 1610 is shaped to be mounted in this void such that air drawn by blower 1602 passes through device 1610 intermediate the vent and the cooling zone. In the present embodiment device 1610 is slidably mounted, and configured for relatively straightforward and time-effective installation. An underlying objective behind this embodiment is for device 1610 to be installable into unit 1601 in a matter of minutes. Various known mounting techniques are used in this regard, such as adhesive mounting brackets. In one embodiment device 1610 simply rests on a lower surface of unit 1601.

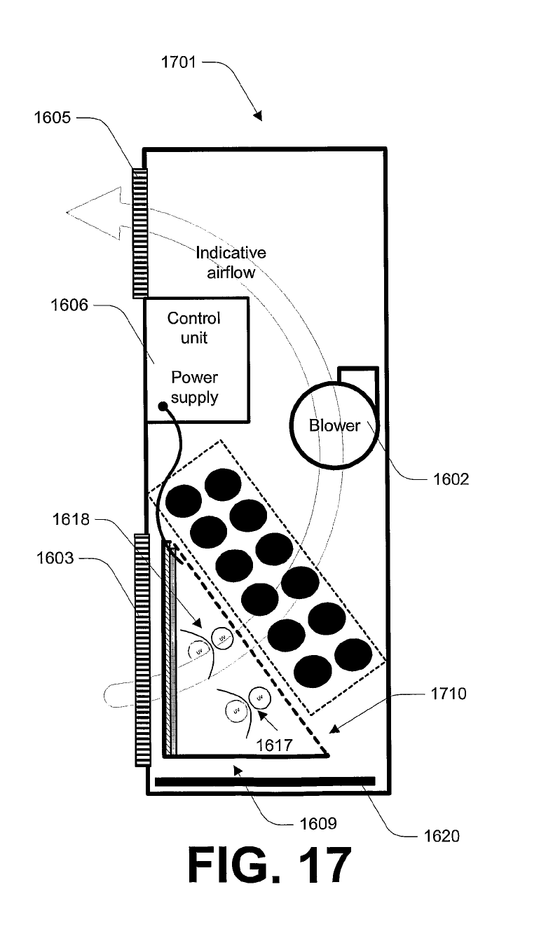

The body of device 1610 is substantially a trapezoidal prism, this conforming to the shape of void 1609. FIG. 17 illustrates an alternate fan coil unit 1701 and an alternative triangular prism shaped device 1710. Generally speaking, appropriate configurations include a wedged shape having a surface configured for substantial conformity with an upstream end of the cooling zone.

Unit 1601 includes an ozone production tube 1612 upstream of an ozone destruction tube 1613, these being separated by a concave reflective baffle 1614. This pair of tubes, in combination with the baffle, defines an ozone production zone 1615 and an ozone destruction zone 1616. The tubes and baffle collectively define a UV processing assembly 1617, and two of these are used in the present embodiment. More or fewer are used in other embodiments, the determining factor primarily being a balancing of device cost considerations, device geometry, and device performance requirements. In other embodiments other assemblies 1617 are used, having varying numbers and configurations of tubes. Likewise, the number of assemblies used in a particular device varies between embodiments.

Other internal surfaces of device 1610 are optionally coated with reflective or catalytic surfaces to enhance ozone production and destruction, as discussed with respect to embodiments further above. A particulate filter 1618 and antimicrobial filter 1619 are also provided inside device 1610. In one embodiment the antimicrobial filter is supported by a wire mesh.

There is a relatively low pressure drop between the upstream and downstream ends of device 1610, typically the order of about 150 Pa. This allows device 1610 to lie in the airflow path of unit 1601 with minimal effect on the power that need be applied to blower 1602. Device 1601 is as such a passive device that is able to provide beneficial air quality management functionalities to an existing unit.

In the present embodiment device 1610 is coupled to central logic 1606 for the purpose of obtaining a supply of power and for the purpose of monitoring operational characteristics of unit 1601. In relation to monitoring, device 1610 is responsive to airflow and other performance characteristics of unit 1601 for varying the power supplied to tubes 1613 and 1614 to provide appropriate ozone production and destruction for the level of air flow at a given time. In particular, the tubes are deactivated when there is no airflow, and/or when unit 1601 is deactivated.

In some embodiments device 1610 is coupled to its own power source and optionally additional monitoring equipment, for example where interfacing with unit 1601 is either relatively difficult or expensive. For example, device 1610 includes an airflow monitor. In one other embodiment device 1610 includes a monitor installable in a connection line intermediate logic unit 1606 and blower 1602 for estimating the amount of power being applied to the blower, thereby to estimate airflow.

To install a device 1610, an installer removes vent 1603, installs one or more mounting brackets (if needed), and slides device 1610 in to place. Connectivity for coupling to unit 1601 is preferably purpose-configured during manufacture of device 1610 such that no complex wiring is required, typically only involving the connection one or a small number of plugs into logic unit 1606 or into an external power supply. An actuator on device 1610 is then activated to power on device 1610, vent 1603 is then replaced, and device 1610 is ready for operation. In one embodiment devise 1610 includes a sensor for ensuring that UV tubes are disabled when vent 1603 is open.

Unit 1601 includes a drip tray 1620, for collecting liquid condensation forming in the cooling zone. By conducting pathogen reduction upstream of the cooling zone, there is there is a significantly reduced risk of pathogens cumulating in that drip tray.

It will be appreciated that device 1610 provides a quick and efficient solution for allowing retrofitting of existing air conditioning units such as unit 1601 to provide odor and pathogen reduction capabilities.

It will be further appreciated that design features discussed in respect of other embodiments discussed herein, such as alternate tube configurations, monitoring equipment and modular design, are optionally implemented in a device such as device 1610 in other embodiments.

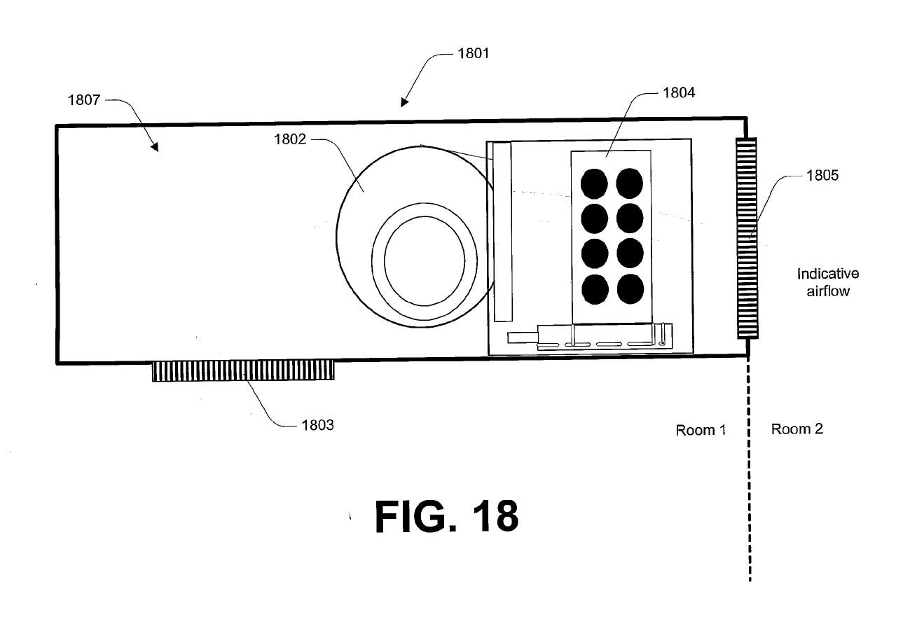

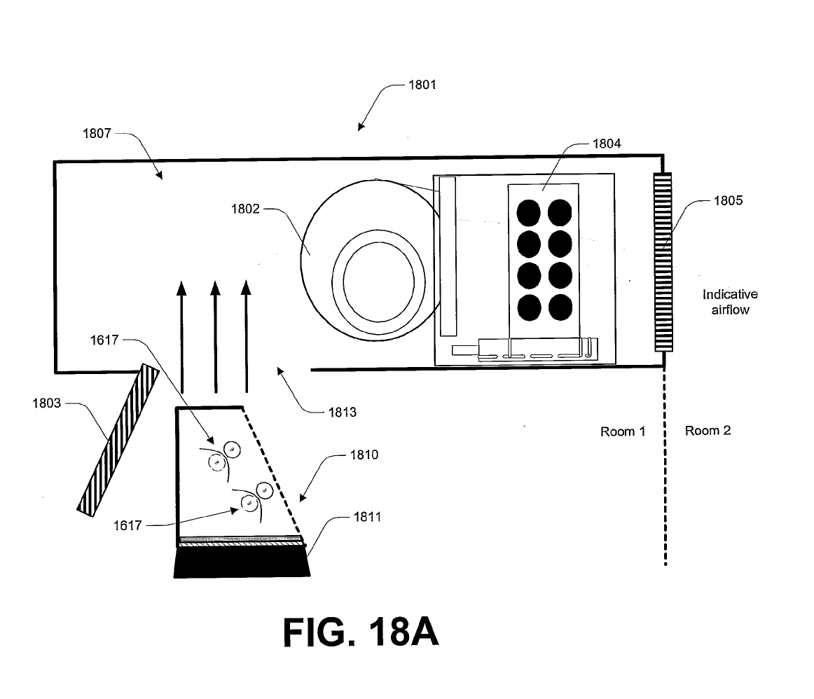

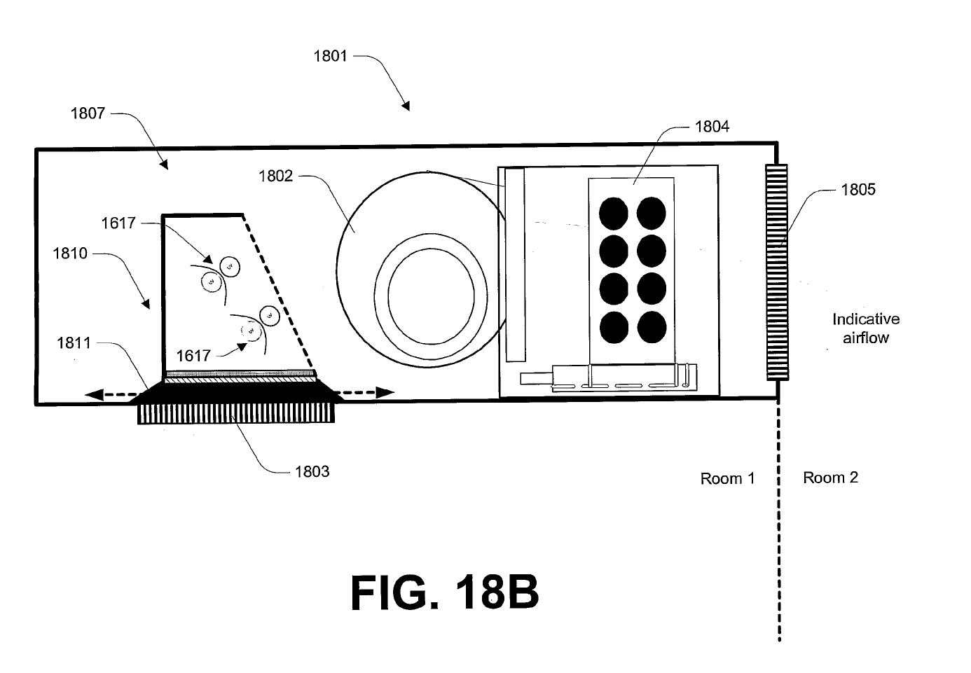

FIG. 18 illustrates a horizontal fan coil air conditioning unit 1801. In overview, a circulation module 1802 is responsible for drawing air through a vent 1803, through a cooling zone 1804, and out of a further vent 1805. In the present embodiment vents 1804 and 1805 are disposed in adjacent rooms. Whilst not in a strict technical sense part of the hardware of unit 1801, for the purpose of the present disclosure roof cavity 1807 is considered to be part of unit 1801.

FIGS. 18A and 18B show a device and method for managing air quality in accordance with further aspects of the present invention, the device taking the form of device 1810. Device 1810 is somewhat similar to device 1610, having a similarly low pressure drop and one or more assemblies 1617. Similar features are designated by corresponding reference numerals.

Device 1810 is contained in a body configured for insertion into cavity 1807. To this end, device 1810 includes an extendable/collapsible base member 1811. Base member 1811 is arranged in a collapsed configuration for insertion through an aperture 1813 concealed by vent 1803, and subsequently moved into an extended configuration once inside the cavity such that device 1811 covers aperture 1813 from above. As such, air drawn by module 1802 is drawn through device 1810 prior to reaching zone 1804. It will again be appreciated that device 1810 provides a passive air quality management device upstream of a cooling zone.

In one embodiment base member 1811 includes a resilient rubber or plastic cone that collapses during the insertion process, and subsequently resiliently expands.

It will be appreciated that device 1810 provides a quick and efficient solution for allowing retrofitting of existing air conditioning units such as unit 1801 to provide odor and pathogen reduction capabilities.

It will be again appreciated that design features discussed in respect of other embodiments discussed herein, such as alternate tube configurations, monitoring equipment and modular design, are optionally implemented in a device such as device 1810 in other embodiments.

Unit 1801 is also generally indicative of various models of air conditioning unit manufactured and sold by Carrier Corporation, including the 42CA Furred-In Horizontal Unit and, 42CE Furred-In Horizontal Unit with plenum.

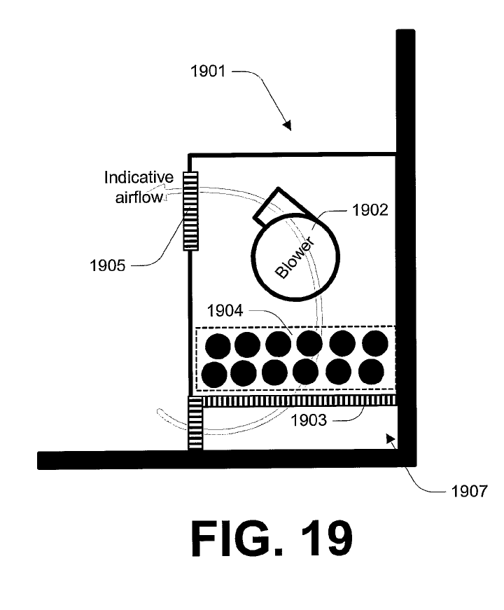

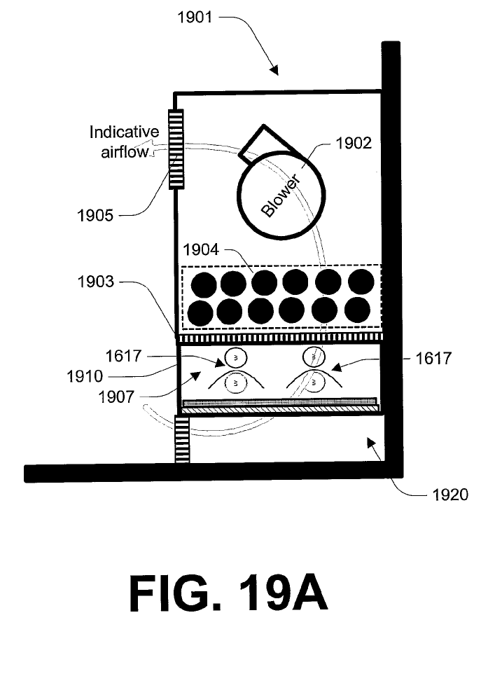

FIG. 19 illustrates another configuration of vertical fan coil air conditioning unit 1901. Units such as this are typically installed in locations such as under window ledges. In overview, a blower 1902 is responsible for drawings air through a vent 1903, through a cooling zone 1904, and out of a further vent 1905. Vent 1903 is disposed above a cavity 1907.

FIG. 19A shows a device for managing air quality in accordance with a further aspect of the present invention, the device taking the form of device 1910. Device 1910 is somewhat similar to device 1610, having a similarly low pressure drop and including one or more assemblies 1617. Once again, similar features are designated by corresponding reference numerals.

Device 1910 is contained in a body configured for insertion into cavity 1907. Additionally, unit 1901 is raised above ground level to provide a replacement cavity 1920 through which air is drawn. In the configuration shown in FIG. 19A, air is drawn by blower 1902 from cavity 1920, then through device 1910, vent 1903, zone 1904, and out through vent 1905.

In one embodiment device 1910 includes a structure slidably mounted as a drawer insertable into and removable from cavity 1907. Removal or partial removal of this drawer provides convenient access to the ozone production and destruction tubes. In another embodiment device 1910 is vertically slidable downwards from cavity 1907 into cavity 1920, then outwardly slidable from cavity 1920 for convenient access. Mounting arrangements to allow such forms of slidable mounting are known.

It will be appreciated that device 1910 provides a quick and efficient solution for allowing retrofitting of existing air conditioning units such as unit 1901 to provide odor and pathogen reduction capabilities.

It will be again further appreciated that design features discussed in respect of other embodiments discussed herein, such as alternate tube configurations, monitoring equipment and modular design, are optionally implemented in a device such as device 1910 in other embodiments.

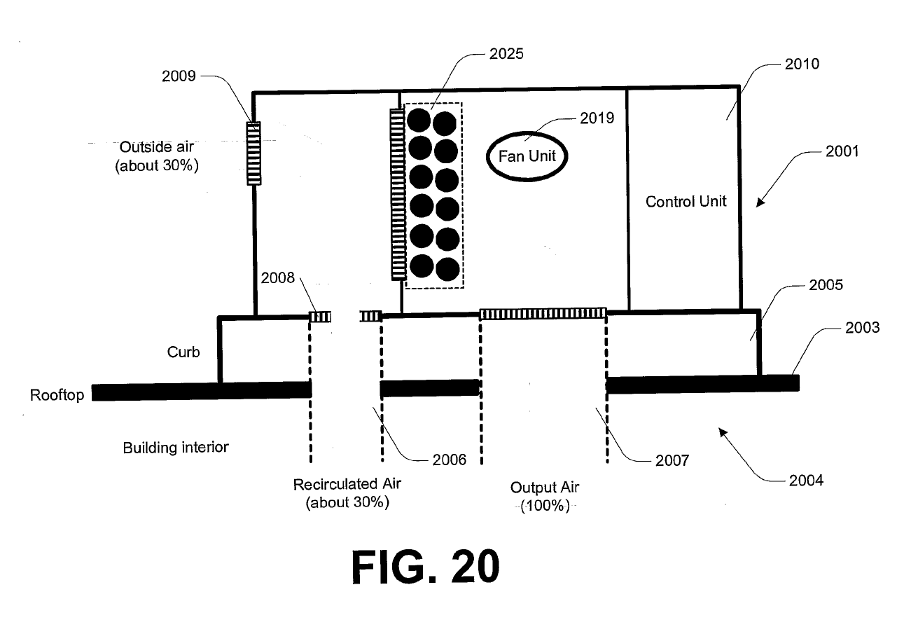

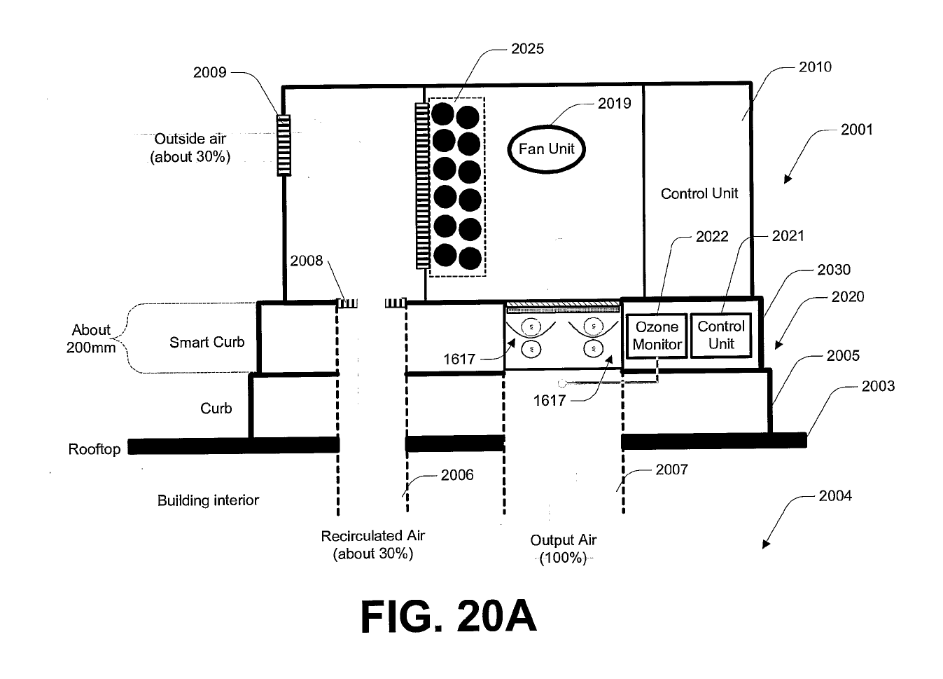

FIG. 20 illustrates a rooftop air processing unit 2001, similar to those manufactured and sold by Trane under their Precedent line. A variety of other entities manufacture and sell similar units. The underlying design premise behind such units is to withdraw recirculated air from a building, combine this in a selected ratio with outside air, perform some cooling, and return the combined air to the building.

Unit 2001 is located on the rooftop 2003 of a building 2004. Specifically, it is supported above rooftop 2003 by a raised curb 2005, which is typically about 200 mm in height. Curbs such as this are widely used to reduce the risk of excess rooftop water leaking into inlet duct 2006 or outlet duct 2007, these ducts extending downwardly into building 2004.

A fan unit 2019 is responsible for drawing recirculated air from a first vent 2008 coupled to duct 2006, and outside air from a second vent 2009. Vents 2008 and 2009 are variably controllable by a central logic unit 2010 for allowing control over the percentages of recirculated air and outside air that are to be returned to building 2004 via duct 2007 after cooling in a cooling zone 2025. Typically a ratio of about 70% recirculated to 30% outside is selected, although it is fairly commonplace to use ranges from 50-50 to 80-20.

It has been suggested previously to place a device for managing air quality upstream of a rooftop unit such as unit 2001. An alternate approach is adopted by device 2020 of FIG. 20A, which is positioned downstream of unit 2001. The underlying rationale is to actively manage the risk of external pathogens being introduced into building 2004. A particular concern in this instance is ozone. An increase in ambient ozone levels has been observed recently in some cities large cities, with several US cities being marked as ozone trouble spots. An ozone content of about 50 parts per billion is considered safe for human respiration, however some large cities are known to have, at least during certain time periods, ambient ozone contents significantly in excess of this level. This means that dangerous levels of ozone may be introduced into a building via the inclusion of outside air into a recirculation system. The inclusion of an ozone destruction zone downstream of an outside air inlet. The present inventors suggest placing a device such as device 2020 including one or more assemblies 1617 downstream of unit 2001 to manage ozone concerns, whilst additionally providing pathogen and odor removal functions.

Device 2020 is again a passive device having ozone production tubes, downstream ozone destruction tubes, a particulate filter, and an antimicrobial filter. In the illustrated embodiment there are two of each types of tube, however in other embodiments there are from one to five of each type of tube, with like tubes being evenly horizontally spaced apart. This is based on a configuration where duct 2007 and corresponding apertures in the curb and rooftop unit are about 1 square meter in cross sectional area. For implementations where there are larger apertures, more tubes are optionally used. For example, about ten, or between five and twenty.

Device 2020 is enabled to adjust its ozone production and ozone destruction ratios to account for levels of ambient ozone, thereby to reduce the risk of ozone being provided to the building. For example, ozone destruction capacity is maintained at a level sufficient to effectively destroy ozone produced in device 2020 as well as ozone received by device 2020 from an upstream source, such as outside.

Device 2020 includes a central logic unit 2021 and ozone monitor 2022. The ozone monitor in this embodiment monitors ozone levels downstream of device 2020 to ensure that ozone discharge does not exceed a threshold level. If this level is exceeded, ozone production is halted or at least reduced. Central logic unit 2021 is responsive to monitor 2022 to determine the ratio of ozone production to ozone destruction that is desired. In one embodiment a timer is implemented such that ozone production is reduced during time periods where a higher outside ozone content is expected, for example during peak hours. In another embodiment a further ozone monitor actively monitors the ozone content of air upstream of device 2020 to determine whether the ration of ozone destruction should be increased or decreased.

Another aspect of the invention provides a structure 2030 for maintaining device 2020. Structure 2030 takes the form of a smart curb, which is a secondary curb installed above curb 2005 for supporting unit 2001. This structure is about 200 mm in height, allowing for a device 2020 of about 150 mm in height. Structure 2030 includes a hollow bottomless slidable drawer for supporting device 2020. This drawer is outwardly slidable to provide convenient access to device 2020. The control unit is configured to deactivate all UV tubes when the drawer is open.

The term “smart curb” describes a supplemental structure built above an existing rooftop curb for supporting a rooftop unit, such as an HVAC unit. A smart curb not only supports, but also provides for air quality management functionalities—hence the “smart” descriptor”. In some embodiments, a tray or drawer, square or horizontal according to the dimensions of the unit it is attached to, is provided on the smart curb for allowing access to internal components, such as UV tubes, antimicrobial filters, sensor controllers, and other control components. In some embodiments a smart curb is an open plenum that allows recirculated air to mix with outside air from the atmosphere in a chamber where pollutants and ozone is controlled emitted and dissipated to provide purified, sterilized and odor free air into an existing duct system inside of a building.

It will be appreciated that device 2020 and structure 2030 allow a simple and convenient retrofit of existing rooftop systems to firstly provide pathogen and odor removal functionality, and secondly to alleviate concerns relating to outside ozone contents.

It will be again further appreciated that design features discussed in respect of other embodiments discussed herein, such as alternate tube configurations, monitoring equipment and modular design, are optionally implemented in a device such as device 2020 in other embodiments.

Noting that some embodiments described above deal with ozone output management, for example device 2020, it is worth considering in detail some of the factors surrounding ozone production and destruction.

The quantity of ozone in discharged air depends on the design parameters a particular embodiment. This is conveniently managed and if necessary modified using a “design curves” approach according to an embodiment of the present invention. Devices such as those discussed above are, prior to end user sale, tested to measure the peak ozone concentration within the unit, and in the discharge air to confirm that it meets specified requirements. A target of 10 ppb ozone with an acceptance of 20 ppb ozone in the discharge air is provided by various devices according to the present invention.

Going into some detail regarding the chemical process used to generate the ozone, when diatomic oxygen (O₂) is irradiated by light with a wavelength below 242 nm (such as 185 nm) it splits into nascent oxygen (a.k.a. oxygen radicals consisting of a single atom). These then react with O₂ to produce ozone (O₃). The inclusion of an optional TiO₂ layer is helpful—whilst has no effect on the production of ozone, it does provide a photocatalyst.

Photocatalytic processes involve photons and a semiconductor catalyst such as titanium dioxide (TiO₂). When the catalyst is irradiated by photons of energy (hv) of at least equal to its band gap energy, electrons are transferred from the valence band to the conductance band. As consequence, charge carriers, positive hole (h+) in the valence band and an electron in the conduction band (e−), appear. These charge carriers (h+, e−) can recombine (reverse process), liberating heat, or be involved in oxidation/reduction reactions which can further trigger thermal or photocatalytic reactions to mineralise the pollutants present in water. Photoinduced oxidation reactions (electron donor) and reduction reactions (electron acceptor) will occur in the valence band and in the conduction band respectively. In the case of TiO₂, light of a wavelength less than 387 nm has an energy above the band gap, and hence produces the charge carriers (h+, e−).

Ozone (O₃) molecules contain three oxygen atoms; molecules of normal (or diatomic) atmospheric oxygen (O₂) contain two. Ozone's germicidal and deodorising properties rely on its tendency to release nascent oxygen (a.k.a. oxygen radicals consisting of a single atom), but the molecule may nonetheless be relatively stable, as witnessed by the ozone layer in the Earth's atmosphere).

In practice, ozone will usually “lose” its extra oxygen in some form of chemical reaction. But reaction rates can be quite slow—air is, after all, roughly 1000 times more “dilute” than most liquids.

However, photochemistry provides one way of significantly increasing reaction rates. Some (not all) molecules will absorb light (often of specific wavelengths), leaving the absorbing molecule in an “excited” or more reactive state. This phenomenon is, for example, the basis of “black and white” film photography.

Ozone has some absorption at visible wavelengths (it is a faintly blue gas), but absorbs more strongly in the ultraviolet. The main emission wavelength from Mercury vapour lamps (254 nanometres) is almost a perfect match for the peak absorption wavelength of ozone. “Normal” (diatomic) oxygen has no significant absorption at 254 nm, and is thus essentially unaffected by such radiation.

At sufficiently high radiation fluxes at this wavelength, pure ozone would be split into diatomic oxygen and nascent oxygen. In these conditions, the nascent oxygen radicals would “pair up” to give diatomic oxygen. Some oxygen radicals would join with diatomic oxygen to re-form ozone, but this ozone would once again be split.

In our case, we are looking at ozone in air—not as a pure gas. If the gas were perfectly dry, the above reaction path would also pertain. But with any significant humidity, ozone will react with water molecules rather than produce oxygen radicals—simply because it is more energetically favourable to do so.

We are unlikely to encounter dry air. Man finds such atmospheres very uncomfortable. In practice, any air that may be treated should always have more than enough water within it for the reaction of ozone with water to predominate.

The photochemical reaction itself could not be simpler. It is:

O₃+H₂O→O₂+H₂O₂

This may not be the end of the story, however. The hydrogen peroxide produced also absorbs at 254 nm (albeit much more weakly), and may be split into two hydroxyl radicals:

H₂O₂→2OH

It may be useful to put these two reactions into perspective. The ability of a molecule to absorb energy (at a specific wavelength) can be represented as its extinction coefficient (ε)—the higher the number, the greater the absorption. At 254 nm, the extinction coefficient for hydrogen peroxide is 18.6 (units are M−1 cm−1). The comparable figure for ozone is 3300. In consequence, the photodecomposition of ozone is general about 1000 times higher than that of hydrogen peroxide.

It would be unfair to represent the two simple chemical equations above as representing the totality of atmospheric chemistry involved here. The combination of O₃, H₂O₂ and OH are collectively known as the “peroxone system”, and these three species are in equilibrium with a number of other (more exotic) free radicals and radical ions.

It might perhaps be thought that the generation of hydrogen peroxide (and of hydroxyl radicals) would be just as undesirable as that of ozone. This is not the case. This state of affairs is due to biology rather than chemistry. H₂O₂ is admittedly a little less oxidising than O₃ (oxidation potentials—in electron volts—are 1.77 and 2.07 respectively), but the main reason is that humans are peculiarly well protected against hydrogen peroxide—because it is an unwanted byproduct of our internal cellular biochemistry, and we have evolved to cope with it. We have a specific enzyme to break down hydrogen peroxide, its name is “catalase”, and it is the fastest acting enzyme yet discovered. By comparison, as a species we seem to have suffered less exposure to ozone during our evolutionary past, and have certainly failed to evolve an equivalent enzyme.

In relation to the decomposition process for common contaminants encountered in commercial buildings, the potential number of malodorous (and sometimes harmful) vapour contaminants in the air is unknown. More than 4,000 constituents have been identified in cigarette smoke alone. In consequence, it will be necessary to group compounds into a relatively small number of classes, reflecting the chemical groups they contain, and giving examples of compounds in these groups.

Whether something smells “bad” or “nice” is not random. Often, the associations of smells are “hard-wired”, as opposed to learned responses. We are generally repelled by odours associated with infection, death, decay, excretion etc., so that we may avoid, where possible, diseases and parasites. Compounds may by chance resemble (in terms of olfaction) those in the list above—if so, the response will be the same, even if the compounds are in no way harmful. Conversely, some compounds may be extremely hazardous, but—if synthetic—smell pleasant; one chemical warfare agent smells of new-mown hay.

Some responses, such as our aversion to body odours—or, indeed, compounds which smell similar to these—may be learned responses. Some such compounds, however—especially the pheromones which we produce—may be detected subliminally, not consciously. We may dislike an environment because of smells we are not, in a sense, even aware of.

Here, four main groups of compounds are considered:
The thiols (a.k.a. mercaptans) and sulphides. Hydrogen sulphide is, of course, the smell of rotten eggs. Methane thiol is released from animal faeces (and in bad breath). One of the main odours of stale sweat is 3-methyl-3-sulphanylhexan-1-ol. Few if any thiols smell pleasant.
The amines. Putrescine (1,4-diaminobutane) and cadaverine (1,5-aminopentane) probably largely speak for themselves. Ammonia-based cleaning agents may sometimes produce compounds of this type if used on fats or greases.
Unsaturated lipids (including alkenes and polycyclics). 3-methyl-2-hexenoic acid is perhaps the archetypal “B.O.” smell. “Benzopyrine” (a chlorinated polyplenol) is one of the more unpleasant constituents of tobacco smoke (and of car exhausts).
Fatty acids (a.k.a. carboxylic acids). Generally not a group of malodorous compounds—although acetic acid (vinegar) is not particularly pleasant—but included here because of a single compound: n-butyric acid (or propane-1-carboxylic acid). Whilst found (and generally tolerated in) parmesan cheese, it is also one of the characteristic odours of vomit.

The action of ozone cannot properly be separated from the various chemical species comprising the “peroxone system”: primarily ozone, hydrogen peroxide and hydroxyl radicals, but also including other free radicals and radical ions.

Thiols and related compounds will be readily oxidised, generally producing —SO₂ groups and thus odourless compounds. Hydrogen sulphide, incidentally, will be oxidised to sulphuric acid—not a perfect product, but the best possible by any chemical process.

Oxidative attack of the double bonds in unsaturated lipids will also be relatively fast, giving harmless (and generally odourless) polyalcohols, in the first instance. Where lipids are chlorinated, these groups are generally unaffected. This group includes the pheromones, such as androstenone and its derivatives in sweat.

Reaction with amide groups may be slower, but will generally proceed by oxidation to odourless nitro compounds.

There are no specific mechanisms related to fatty acids—the active groups are, essentially, already oxidised.

However, almost all the compounds discussed above involve aliphatic or aromatic compounds with carbon-carbon bonds. Particularly where there are reactive groups such as the —COOH groups of fatty acids (or the —OH groups of alcohols), these regions of molecules may be broken down by ozone and other members of the peroxone system to give compounds no more toxic (or odiferous) than water and carbon dioxide. Polycyclic compounds (including the carcinogenic “benzopyrine”) may similarly be attacked and converted to harmless (and generally odourless) oxidation products.

It is generally accepted that ozone, at worst, produces many fewer toxic compounds than it produces. Clearly, where breakdown products may be only water and carbon dioxide, there can be no concerns. In terms of the attack of thiol- and amine-containing compounds, there appear to be no reports in the available literature of hazardous breakdown products. There are reports of potentially toxic oxidation products (e.g. bromates from dissolved bromides) in aqueous systems, but not in air treatment. By comparison, examples of toxic oxidation products from chlorine-based agents are commonplace.

Hydrogen sulphide (as toxic as cyanide gas) is perhaps an unlikely pollutant to encounter, and so perhaps is carbon monoxide in these catalytic days, but it may be worth noting that ozone will convert this to harmless carbon dioxide.

Generally, ozone (and the other members of the peroxone system) will generally—by various oxidative processes—destroy the chemical groups which exist in malodorous compounds (i.e. those the human nose detects and find unpleasant). It will also tend to convert potentially toxic compounds into non-toxic ones. The proven germicidal properties of ozone are not, however, discussed herein.

Whilst embodiments have been described by reference to air processing units having various components in their respective treatment paths, it will be appreciated that other embodiments make use of other combinations and permutations of these and other components.

Unless specifically stated otherwise, as apparent from the following discussions, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included. Thus, one example is a typical processing system that includes one or more processors. Each processor may include one or more of a CPU, a graphics processing unit, and a programmable DSP unit. The processing system further may include a memory subsystem including main RAM and/or a static RAM, and/or ROM. A bus subsystem may be included for communicating between the components. The processing system further may be a distributed processing system with processors coupled by a network. If the processing system requires a display, such a display may be included, e.g., a liquid crystal display (LCD) or a cathode ray tube (CRT) display. If manual data entry is required, the processing system also includes an input device such as one or more of an alphanumeric input unit such as a keyboard, a pointing control device such as a mouse, and so forth. The term memory unit as used herein, if clear from the context and unless explicitly stated otherwise, also encompasses a storage system such as a disk drive unit. The processing system in some configurations may include a sound output device, and a network interface device. The memory subsystem thus includes a computer-readable carrier medium that carries computer-readable code (e.g., software) including a set of instructions to cause performing, when executed by one or more processors, one of more of the methods described herein. Note that when the method includes several elements, e.g., several steps, no ordering of such elements is implied, unless specifically stated. The software may reside in the hard disk, or may also reside, completely or at least partially, within the RAM and/or within the processor during execution thereof by the computer system. Thus, the memory and the processor also constitute computer-readable carrier medium carrying computer-readable code.

Furthermore, a computer-readable carrier medium may form, or be includes in a computer program product.

In alternative embodiments, the one or more processors operate as a standalone device or may be connected, e.g., networked to other processor(s), in a networked deployment, the one or more processors may operate in the capacity of a server or a client machine in server-client network environment, or as a peer machine in a peer-to-peer or distributed network environment. The one or more processors may form a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine.

Note that while some diagrams only show a single processor and a single memory that carries the computer-readable code, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein.

Thus, one embodiment of each of the methods described herein is in the form of a computer-readable carrier medium carrying a set of instructions, e.g., a computer program that are for execution on one or more processors, e.g., one or more processors that are part of building management system. Thus, as will be appreciated by those skilled in the art, embodiments of the present invention may be embodied as a method, an apparatus such as a special purpose apparatus, an apparatus such as a data processing system, or a computer-readable carrier medium, e.g., a computer program product. The computer-readable carrier medium carries computer readable code including a set of instructions that when executed on one or more processors cause the processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (e.g., a computer program product on a computer-readable storage medium) carrying computer-readable program code embodied in the medium.

The software may further be transmitted or received over a network via a network interface device. While the carrier medium is shown in an exemplary embodiment to be a single medium, the term “carrier medium” should be taken to include a single medium or multiple media (e.g., a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media. Non-volatile media includes, for example, optical, magnetic disks, and magneto-optical disks. Volatile media includes dynamic memory, such as main memory. Transmission media includes coaxial cables, copper wire and fiber optics, including the wires that comprise a bus subsystem. Transmission media also may also take the form of acoustic or light waves, such as those generated during radio wave and infrared data communications. For example, the term “carrier medium” shall accordingly be taken to included, but not be limited to, solid-state memories, a computer product embodied in optical and magnetic media, a medium bearing a propagated signal detectable by at least one processor of one or more processors and representing a set of instructions that when executed implement a method, a carrier wave bearing a propagated signal detectable by at least one processor of the one or more processors and representing the set of instructions a propagated signal and representing the set of instructions, and a transmission medium in a network bearing a propagated signal detectable by at least one processor of the one or more processors and representing the set of instructions.

It will be understood that the steps of methods discussed are performed in one embodiment by an appropriate processor (or processors) of a processing (i.e., computer) system executing instructions (computer-readable code) stored in storage. It will also be understood that the invention is not limited to any particular implementation or programming technique and that the invention may be implemented using any appropriate techniques for implementing the functionality described herein. The invention is not limited to any particular programming language or operating system.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

All publications, patents, and patent applications cited herein are hereby incorporated by reference.

Any discussion of prior art in this specification should in no way be considered an admission that such prior art is widely known, is publicly known, or forms part of the general knowledge in the field.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

Similarly, it is to be noticed that the term coupled, when used in the claims, should not be interpreted as being limitative to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other. For example, in the context of airflow, where an outlet of A is coupled to an inlet of B it may be that one or more additional devices are provided between the outlet of A and the inlet of B

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A module for use with a system for managing air quality, wherein the system includes an inlet for receiving feed air and an outlet downstream of the inlet for passing the feed air, the module including:
a body removably mountable to the system such that the feed air travels through the module intermediate the inlet and the outlet;
one or more UV tubes in the body for emitting UV light at an ozone production wavelength and/or an ozone destruction wavelength.

2. A module according to claim 1 wherein at least one of the UV tubes is configured to be disposed normal to the direction of airflow when the body is mounted to the system.

3. A module according to claim 1 including a plurality of vertically spaced UV tubes.

4. A module according to claim 1 wherein the one or more UV tubes include at least one dual purpose UV tube having a first portion coated to emit UV light at an ozone production wavelength and a second portion coated to emit UV light at an ozone destruction wavelength.

5. A module according to claim 4 wherein the portions are defined such that less UV light is emitted at the ozone production wavelength than at the ozone destruction wavelength.

6. A module according to claim 4 wherein the first and second portions are circumferential portions.

7. A module according to claim 4 wherein the first and second portions are longitudinal portions.

8. A module according to claim 1 wherein the one or more UV tubes include at least one ozone production tube for emitting UV light at an ozone production wavelength and at least one ozone destruction tube for emitting UV light at an ozone destruction wavelength.

9. A module according to claim 8 including a reflector intermediate the at least one ozone production tube and the at least one ozone destruction tube thereby to define a ozone production zone and an ozone destruction zone, wherein UV light from the at least one ozone destruction tube is substantially absent in the ozone production zone and UV light from the at least one ozone production tube is substantially absent in the ozone destruction zone.

10. A module according to claim 9 wherein the reflector is curved about the at least one ozone production tube.

11. A module according to claim 1 wherein the system is defined by an air conditioning unit having a cooling zone, and wherein the body is removably mountable intermediate the inlet and the cooling zone.

12. A module according to claim 11 wherein the air conditioning unit is a vertical furred-in stack type fan coil air conditioning unit.

13. A module according to claim 1 wherein the one or more UV tubes are replaceably mounted in the body.

14. A method a modifying an air conditioning unit having an inlet upstream of a cooling zone, the method including the step of disposing a module according to claim 1 intermediate the inlet and the cooling zone.

15. A system for managing air quality, the system including:
a frame for defining:
an inlet for receiving feed air from a unidirectional air passage at a flow rate;
a module housing region for removably housing one or more air processing modules, the module housing region being configured for removably housing at least one UV module for containing one or more UV tubes that emit UV light at an ozone production wavelength and/or an ozone destruction wavelength; and
an outlet coupled to the treatment path for receiving the feed air from the treatment path and passing the feed air to the unidirectional air passage at a location downstream of the input; and
a power supply for providing power to at least one module upon housing of that module in the module housing region.

16. A system according to claim 15 wherein the module housing region is configured for removably housing at least one antimicrobial filter module.

17. A system according to claim 15 wherein the module housing region is configured for removably housing a plurality of UV modules.

18. A system according to claim 17 wherein at least two of the plurality of UV modules are vertically stacked with respect to one another.

19. A module according to claim 17 wherein at least one of the UV tubes is configured to be disposed normal to the direction of airflow between the inlet and outlet.

20. A system for managing air quality including:
an inlet for receiving feed air from a unidirectional air passage at a flow rate;
a treatment path coupled to the inlet for receiving, processing and passing the feed air to remove one or more air contaminants;
an outlet coupled to the treatment path for receiving the feed air from the treatment path and passing the feed air to the unidirectional air passage at a location downstream of the input, wherein the pressure drop between the inlet and outlet is less than a predetermined value for a given flow rate.
